(19) United States
(12) Patent Application Publication
(10) Pub. No.: US 2023/0124917 A1
(43) Pub. Date: Apr. 20, 2023

(54) SYSTEMS AND METHODS FOR MONITORING AND IDENTIFYING PHYSIOLOGICAL IMPACT EVENTS

(71) Applicant: Central Intelligence Agency, Washington, DC (US)

(72) Inventor: Michael P. Gordon, Middletown, MD (US)

(73) Assignee: Central Intelligence Agency, Washington, DC (US)

(21) Appl. No.: 17/966,181

(22) Filed: Oct. 14, 2022

Related U.S. Application Data
(60) Provisional application No. 63/256,352, filed on Oct. 15, 2021.

(51) Int. Cl. G16H 50/80

(52) U.S. Cl. CPC G16H 50/80; A61B 5/7275

(57) ABSTRACT

The present subject matter addresses a process and testing procedures for establishing a baseline medical status and tracking changes to an individual's immune system with exposure to impact events over time. In particular, the processes described herein establish a baseline medical status for the individual and, through periodic or otherwise initiated medical sampling, map the changes and development of an individual's immune system using biomarkers for exposures. These biomarkers are cataloged as part of an individual medical profile and can assist in bio-surveillance efforts to identify biomarkers for global under-reported and under-researched pathogens through the study of individuals originating from or visiting highly infectious areas. In particular, the present subject matter relates to a method for the diagnosis of an impact event to physiology as well as a system for implementing such analysis and providing treatment options and alerts.

### CROSS-REFERENCE TO RELATED APPLICATIONS

This application claims priority to U.S. Application No. 63/256,352, filed Oct. 15, 2021, which is herein incorporated by reference in its entirety for all purposes.

### GOVERNMENT INTEREST STATEMENT

The United States Government has rights in this invention pursuant to the employer-employee relationship of the Government to at least one inventor.

### BACKGROUND

Physical injury, tissue damage, inflammation, pathogen exposure (including viruses, bacteria, fungi, protozoa, and worms), poison, parasites, infection, disease, traumatic stress, airborne or drinking water pollutants, radiological exposure, radio frequency signal, and other hazardous health exposures (herein referred to as “pathogens” or “exposures”) are extremely abundant and highly present throughout the world. However, the effects of the diverse population of pathogens on short-term and long-term health is not well understood and no bio-surveillance or systematic process exists to provide early warning of epidemic or pandemic health conditions across diverse populations. Pathogen exposure often presents lingering and/or latent physical effects long after a patient has seemingly recovered from the initial impact. Currently, most pathogenic exposures are only identified at the point of care for individuals who have developed symptoms significant enough to warrant professional health care intervention. Accordingly, tracking symptomatic exposure timelines, the spread of the pathogen and treatment are often performed later-in-time and are dependent upon the exposed individual reporting exposure to medical professionals and/or seeking medical assistance.

To identify the effects of pathogens or other physiological impact events, epigenetic sequencing can be used to identify genetic and epigenetic modifications that cannot be attributed to changes in the primary physical DNA genome sequence. These alterations can include DNA interactions with proteins as well as biochemical modifications of nucleic acid bases. The intricacies of these alterations require specific sequencing, characterization pipelines and complex data analysis thereby rendering real-time analysis impractical. Accordingly, these bioinformatic processes require significant memory storage and computing power to perform quality control measures and assemble the data into sequence data strings for evaluation. Subsequent comparisons of the data for updated health evaluation require the same large investment in money, time, memory, computational power and data assembly.

### SUMMARY OF THE INVENTION

Creating, tracking and cataloging an individual's baseline medical status (BMS), and/or those of the individuals in a population over time is extremely valuable to the diagnosis of impact events, such as rare diseases, for individuals long after initial exposure. Further, a priori tracking and cataloging of an individual or a population's biomarker and BMS over time will contribute greatly to identifying unique biomarkers correlating to rare diseases or other physiological effects thereby improving early detection and treatment options. Unlike existing computationally difficult and unrealistic methods, applying anatomical modularization and modified hash function approaches to sequencing enables rapid epigenetic analysis and correlation to anatomic BMS. This enables expedited health checks with less memory and/or overhead processing requirements and allows for additional applications such as real-time assessment and treatment as well as bio-surveillance tracking of individuals traveling to and from areas having exposure risks. This enables rapid preventative actions to be taken to slow or halt the spread of diseases in an increasingly global world.

The present subject matter relates generally to illness and disease identification through establishment of a BMS and identifying changes to the BMS through exposures that cause symptomatic illness or disease. The systems and methods described herein also provide for a global bio-surveillance early indications warning system of potential epidemic or pandemic events. Accordingly, described herein is a method of identifying a disease that includes identifying an immune profile in an individual, monitoring the individual for changes in the immune profile and correlating changes in the immune profile with a disease.

Also described herein is a system for predicting a disease that includes a storage medium configured with a database of biomarkers for an individual, including baseline entries of the biomarkers for the individual, and a processor connected to the storage medium, wherein the processor is configured to compare the biomarkers to a correlation table of biomarkers and diseases.

Further described herein is that the database of the system for predicting a disease includes entries for multiple individuals. Additionally, the processer of the system for predicting a disease is configured with an artificial intelligence and machine learning algorithm configured to monitor the database for similar disease biomarkers in more than two individuals.

The foregoing paragraphs have been provided by way of general introduction and are not intended to limit the scope of the following claims. Therefore, the above summary is not intended to be an exhaustive discussion of all the features or embodiments of the present disclosure. A more detailed description of the features and embodiments of the present disclosure will be described in the detailed description section.

### BRIEF DESCRIPTION OF THE DRAWINGS

A more complete appreciation of the present subject matter and many of the attendant advantages thereof will be readily obtained as the same becomes better understood by reference to the following detailed description when considered in connection with the accompanying drawings, wherein:

FIG. 1 is a diagram of an exemplary environment for a physiological assessment system;

FIG. 2 illustrates an exemplary flowchart of a physiological monitoring process;

FIG. 3 illustrates a Venn diagram of physiological monitoring processes;

FIG. 4 illustrates an exemplary flowchart of a baseline medical status (BMS) generation process;

FIG. 5 illustrates an exemplary process utilizing cell free DNA (cfDNA);

FIG. 6 illustrates additional details of the exemplary process of FIG. 5;

FIG. 7 illustrates one example of how an individual's Major Histocompatibility Complex (MHC) is combined with the Immune Signature and a Respiratory Pathogen Panel (RPP) to establish, and to subsequently compare with, the individual's BMS;

FIG. 8 illustrates an exemplary flowchart of an epigenetic sequencing process;

FIG. 9 illustrates an exemplary modularized breakdown of anatomical systems and corresponding representative MHVBs for each segment;

FIG. 10 provides an example of how DNA is modularized into anatomical segments and then is converted into Small Hash Values (SHVs);

FIG. 11 provides an example of DNA that has been modularized;

FIG. 12 illustrates an exemplary process for mapping the collected sample to the anatomic organ/system;

FIG. 13 illustrates subsequent specific epigenetic testing according to one example;

FIG. 14 illustrates exemplary use of precision data to establish an individual's personal baseline for health tracking;

FIG. 15 illustrates an exemplary analysis stage of the process through which various assays are combined to aid in the diagnosis of a condition caused by exposure (vice due to genetic inheritance);

FIG. 16 illustrates the biomarker contents of a database that represent an impact correlation table according to one example;

FIG. 17 illustrates exemplary cataloged BMS data of an individual over time;

FIG. 18 illustrates an exemplary biomarker impact correlation table;

FIG. 19 illustrates an exemplary detailed biomarker impact correlation table;

FIG. 20 illustrates an exemplary correlation between initiator and condition;

FIG. 21 illustrates an exemplary process for creating an epigenetic sequence baseline hash value;

FIG. 22 illustrates an exemplary graphical user interface for interaction with the physiological assessment system;

FIGS. 23A and 23B illustrates various aspects of an exemplary architecture implementing the system for physiological assessment; and

FIGS. 23C and 23D illustrate an exemplary server interface for connecting user computing devices within the system for physiological assessment.

Like reference symbols in various drawings indicate like elements.

### DETAILED DESCRIPTION

As used herein “substantially”, “relatively”, “generally”, “about”, and “approximately” are relative modifiers intended to indicate permissible variation from the characteristic so modified. They are not intended to be limited to the absolute value or characteristic which it modifies but rather approaching or approximating such a physical or

functional characteristic.

In the detailed description, references to “one embodiment”, “an embodiment”, or “in embodiments” mean that the feature being referred to is included in at least one embodiment of the present subject matter. Moreover, separate references to “one embodiment”, “an embodiment”, or “in embodiments” do not necessarily refer to the same embodiment; however, neither are such embodiments mutually exclusive, unless so stated, and except as will be readily apparent to those skilled in the art. Thus, the present subject matter can include any variety of combinations and/or integrations of the embodiments described herein.

The terminology used herein is for the purpose of describing particular embodiments only and is not intended to be limiting of the present subject matter. As used herein, the singular forms, “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of at least one other feature, integer, step, operation, element, component, and/or groups thereof.

It will be appreciated that as used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus.

It will also be appreciated that as used herein, any reference to a range of values is intended to encompass every value within that range, including the endpoints of said ranges, unless expressly stated to the contrary.

As described further herein, aspects of the present subject matter are described below with reference to flowchart illustrations and/or block diagrams of methods, apparatus (systems) and non-transitory computer-readable mediums according to embodiments of the present subject matter. It will be understood that each block of the flowchart illustrations and/or block diagrams, and combinations of blocks in the flowchart illustrations and/or block diagrams, can be implemented by computer program instructions. These computer program instructions may be provided to a processor of a special purpose computer, or other programmable data processing apparatus to produce a machine, such that the instructions, which execute with the processor of the computer or other programmable data processing apparatus, create means for implementing the functions/acts specified in the flowchart and/or block diagram block or blocks.

These computer program instructions may also be stored in a computer readable medium that can direct a computer, an operating system, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, a processor, other programmable data processing apparatus, or other devices to cause a series of operational steps to be performed on the computer, the processor, or other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present subject matter. In this regard, each block in the flowchart or block diagrams may represent a module, segment, or portion of code, which comprises at least one executable instruction for implementing the specified logical function(s).

It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Referring now to the drawings, wherein like reference numerals designate identical or corresponding parts throughout the several views, the following description relates to systems and methods for monitoring and identifying physiological impact events.

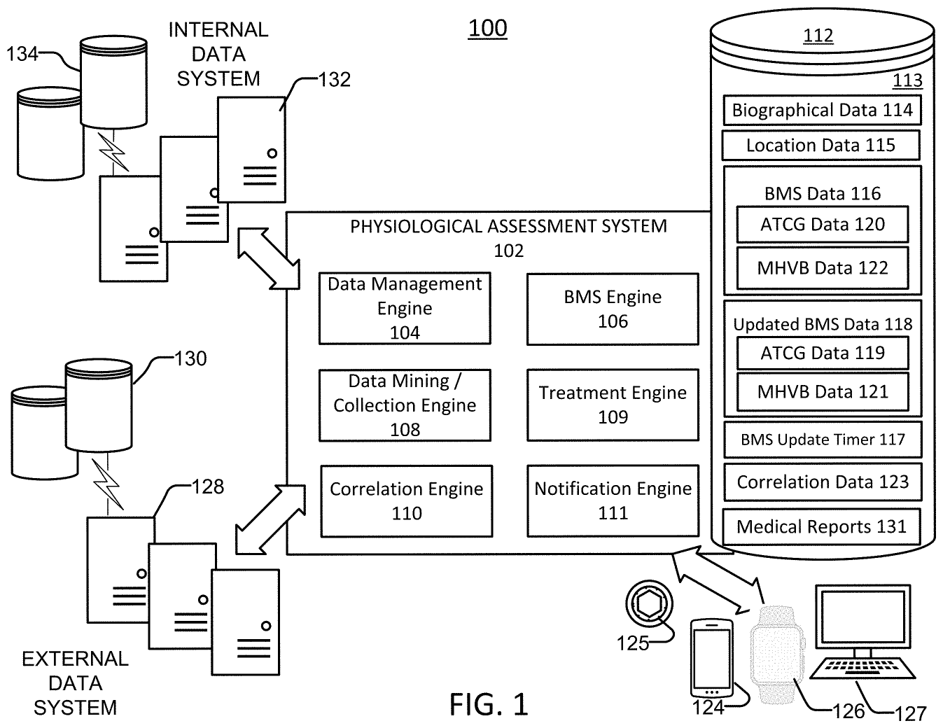

As illustrated in FIG. 1, an environment 100 includes a physiological assessment system 102 connected to one or more databases 112 and further being connected to a plurality of devices or systems including, but not limited to, mobile devices 124, wearable devices 125, 126 and computing devices 127 of an individual and/or other users (i.e. medical practitioners), external data systems having one or more servers 128 connected to one or more databases 130, and internal data systems having one or more servers 132 connected to one or more databases 134. The database 112 may be any type of database and/or memory, either local or not local, such as long-term or short-term storage as would be understood by one of ordinary skill in the art. The data shown in database 112 in one example constitutes a snapshot of data being used by the physiological assessment system 102 at any given time to execute the processes described herein based on data from one or more of the devices 124-127 and/or data mined by the data mining/collection engine 108 from one or both of the external data system databases 130 or internal system databases 134 via servers 128 and 132, respectively, and managed by the data management engine 104. The physiological assessment system 102 further includes a baseline medical status (BMS) engine 106 for generating BMS data 116, 118, a correlation engine 110 for identifying correlations between existing BMS data 116 and periodically updated BMS data 118 or updated BMS data 118 arising out of individual exposure to impact events, a treatment engine 109 for generating treatment information and reports based on data generated by the correlation engine 110 and a notification engine 111 for providing notifications regarding particular correlations, treatments and/or reports to the individual or other user. The interactions between devices and the physiological assessment system 102 can be performed either through direct connection or wirelessly as would be understood by one of ordinary skill in the art.

As further described herein, the external data system can represent one or more third-party systems with respect to the physiological assessment system 102, accessible via the Internet or other external networks, storing information relevant to the functionality of the physiological assessment system 102. External data can be retrieved before and/or during execution by the data mining/collection engine 108 which can access, for example, the data of the external systems via general web-crawling and through use one or more internal or external Application Programming Interfaces (APIs) as would be understood by one of ordinary skill in the art. Once retrieved by the data mining/collection engine 108, the data management engine 104 can save collected data to internal databases 134 and/or provide to the database 112 a particular instance of the retrieved data, such as biographical data 114 and location data 115, for a particular individual. This data can then be used by the physiological assessment engine 102 to perform the methods and provide the functionally described herein.

The internal data systems represent systems storing data local to the physiological assessment system 102 in terms of limited access to third parties of the data stored in databases 134. In one example, the one or more servers 132 and one or more databases 134 process and host data that is used by the physiological assessment system 102 for executing the processes and providing the functionality described herein. As such, the internal databases 134 can store data pertinent to individuals such as data making up an individual medical profile 113. When needed by the physiological assessment system 102, this data can be retrieved by a data mining/collection engine 108 and once retrieved, the data management engine 104 will store a particular instance of the data in the database 112 for the particular individual to be processed by one or more engines of the physiological assessment system 102.

As described further herein and in one example, the wearable device 125 can be a reusable or disposable patch configured to monitor for individual impact events and the wearable device 126 may be any electronic wearable such as a smartwatch or, for example, a device that can be worn in any bodily location that produces consistent perspiration and skin microbiome access. In one example, the wearable 125 is an adhesive patch that may cooperate with field testing kits that provide rapid results for certain blood, saliva, sweat, stool, urine, or other biological samples that may be readily obtained in a non-sterile environment. The field testing kits may include any technology for rapid testing, including but not limited to reactive testing strips with an indicator of the presence or absence of a biomarker, small volume testing strips with a reader such as a diabetes testing kit or a microfluidics “lab-on-a-chip”, or a continuous monitor, such as the technology of a continuous glucose monitor that continuously reads a biological sample and locally stores the data and/or transmits the data to a reader including but not limited to an application program such as a mobile app or Web app or a radio frequency identification (RFID) system. If a field-testing kit is used, the reader can interact with physiological assessment system 102 directly or indirectly, for example by inputting the data thereto or by being connected to the physiological assessment system 102 directly or via any communication network, such as, for example, a cellular or satellite network or the internet.

The wearable device 125 may also be a micro-needling patch that can provide medication to the individual via the patch itself as described in “Microneedles: A smart approach and increasing potential for transdermal drug delivery system,” Waghule et al., Biomedicine & Pharmacotherapy Volume 109, January 2019 (pages 124-1258), the entirety of which is herein incorporated by reference. Thus, medication or other supplements can be provided via microneedles which go through the skin barrier to create tunnels for transmission to the individual. The length of the needle may vary in different embodiments based on comfort considerations or based on an evaluation of the BMS data 116 of the individual as to whether the needles need to penetrate into the dermis or just enough to go past the dead stratum corneum. A variety of micro-needles are contemplated herein for use with the wearable device 125 such as solid steel microneedles, hollow microneedles, hydrogel and dissolving microneedles as would be understood by one of ordinary skill in the art. The wearable device 125 microneedle patch in certain embodiments may contain a microprocessor and receiver which activates the wearable device 125 to induce medication to an individual in response to signals received from the physiological assessment system 102 notification engine 111 based on an assessment of the individual's BMS data 116 by the BMS engine 106.

The wearable 125 can be worn in many different locations on the body and is not restricted to the wrist. The wearable provides notification of anomalous biomarker(s) detection via one or more alert methods including but not limited to (1) color detection using a spectrometer device such as Raman to detect the makers on the adhesive patch, (2) reactive visual dye colors on the wearable patch, (3) electronic alerting sent via wireless signal to other devices 124, 126, 127, and (4) direct alerting on the electronic device using visual, audio, and/or haptic feedback.

The devices 124-127 can be controlled by one or more users and can have mobile application software installed thereon for interfacing with the physiological assessment system 102. The devices 124-127 can have local application software installed thereon for interfacing with the physiological assessment system 102 or can interface with the physiological assessment system 102 via a web-based platform as would be understood by one of ordinary skill in the art. Further, in one example, the physiological assessment system 102 software itself, with or without the database 112, can be installed entirely on one or more of the devices 124-127. In other words, the software installed on the devices 124-127 can include programming for the entire physiological assessment system 102 such that the processes described herein are performed entirely on one or more of the devices 124-127. However, as illustrated in FIG. 1 and for explanation forthwith, it will be assumed that the physiological assessment system 102 is separate from the devices 124-127 and performs the methods described herein based in part on information received from the devices 124-127 via their application interface and databases 130, 134. The physiological assessment system 102 notification engine 111 can then return results of the processes described herein for analysis and presentation to one or more users of the devices 124-127.

The series of connections between the aforementioned devices in the environment 100 can, via the physiological assessment system 102, be used to establish a BMS for an individual and monitor the BMS for any changes over time. The changes over time to the BMS of one individual, with or without additional data, can be analyzed by the physiological assessment system 102 to provide reports, any applicable treatment options and potential medical alerts to an individual or third party for immediate or future actions. Multiple BMS data for a plurality of individuals, with or without additional data, can be utilized by the physiological assessment system 102 to provide aggregate reports regarding potential wide-spread pathogenic impact as well as corresponding alerts based thereon. In the context of the subject matter described herein, the term “individual” may be used interchangeably as describing a human, group of humans, animal or a group of animals, or another type of living organism.

The physiological assessment system 102 implements one or more processes to establish an individual's BMS and periodically, or as a result of exposure to an impact event, tracks changes to the individual's BMS over time and life cycle. In this context, an “impact event” is anything that impacts the BMS of the individual including, but not limited to, pathogens and exposures (i.e. physical injury, tissue damage, inflammation, pathogen exposure (including viruses, bacteria, fungi, protozoa, and worms), poison, parasites, infection, disease, traumatic stress, airborne or drinking water pollutants, radiological exposure, radio frequency signal exposure and other hazardous health exposures). More specifically, using biomarkers and/or determined through testing and/or data from wearable devices 125,126, the physiological assessment system 102 establishes a BMS for an individual and through later-in-time medical sampling maps any changes and development of an individual's system with respect to any alterations in their BMS.

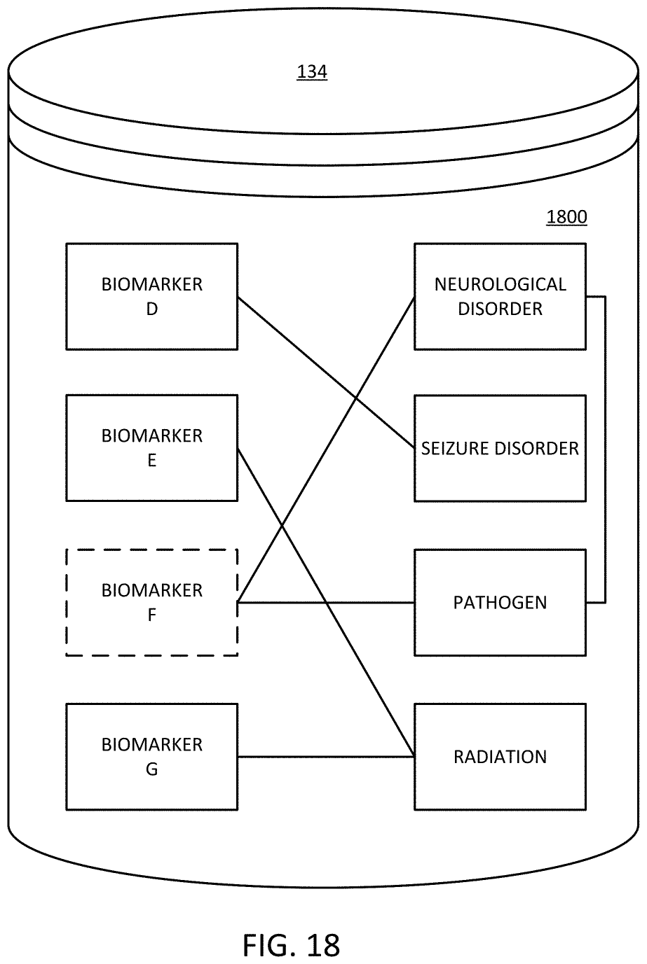

Biomarker information refers to the measurement of potentially health-relevant biomolecules such as nucleic acids, proteins, antibodies, enzymes and/or lipids that are obtained non-invasively or minimally invasively from biofluids such as blood, saliva, sweat and/or urine of the individual. FIG. 18 illustrates the biomarker contents of a database 134 (including assay type as well as examples of indicators and/or episode causes) that represent a biomarker impact correlation table 1800 according to one example based on prototyping markers, such as sweat inflammation markers. These biomarkers are cataloged in one or more databases 134 specifically with respect to particular individuals as BMS data 116, 118 as well as broadly with individual or cross-correlations to potential pathogens, diseases, disorders, etc. In some instances, for example, the presence of such biomarkers can provide early detection information as such biomarkers that are potentially carcinogenic and/or cause neurological issues. In other instances, the absence of certain biomarkers may represent a risk to the individual which should be reported by the physiological assessment system 102 notification engine 111.

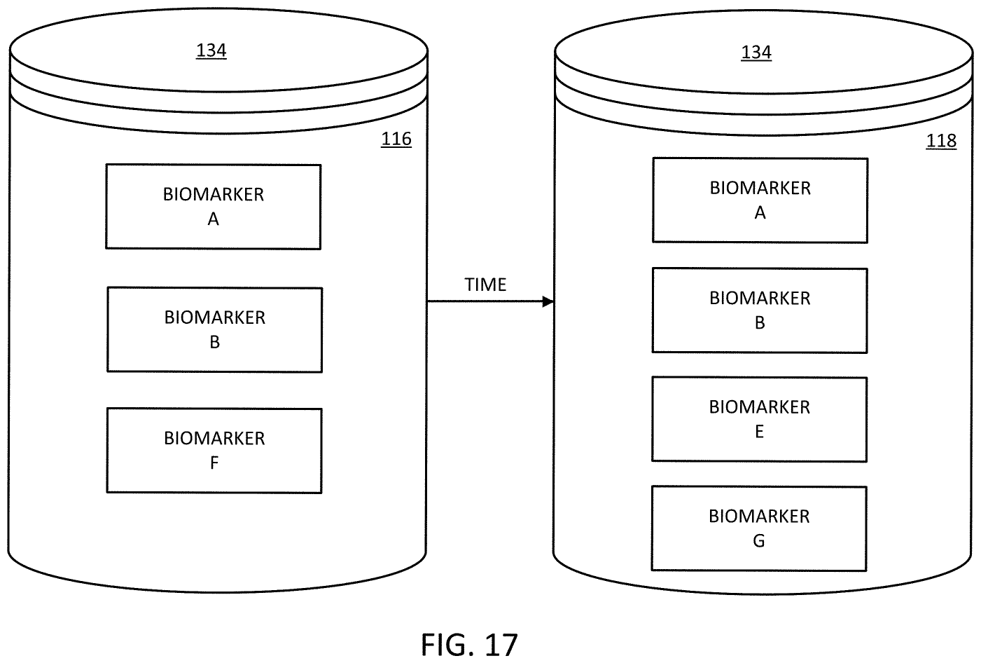

FIG. 17 illustrates the cataloged BMS data 116 of an individual derived from an initial analyses and mapping of individual biomarkers as well updated BMS data 118 which is indicative of changes to BMS biomarker makeup over time. As discussed further herein, FIG. 17 will be used as an example when describing the functionality of the physiological assessment system 102.

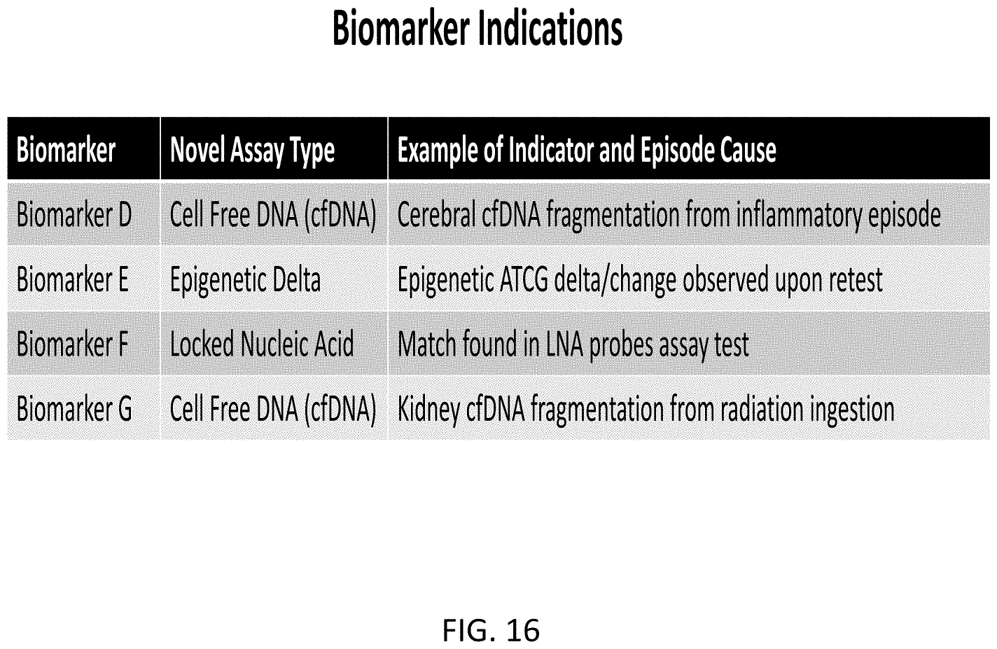

FIG. 18 illustrates an example of a biomarker impact correlation table 1800 stored in databases 134 (and for example having the data illustrated in FIG. 16) for a plurality of biomarkers that is referenced by the physiological assessment system 102 after identifying changes that have taken place to an individual's BMS (such as those illustrated in FIG. 17) and for reporting alerts and treatment options. As illustrated in FIG. 18, there are a variety of established correlations between certain biomarkers (or the absence thereof) and physiological conditions. For example, biomarker D may be found in individuals that have seizure disorders, biomarkers E and G may indicate an exposure to radiation and the absence of biomarker F may indicate an exposure to a pathogen which causes a neurological disorder.

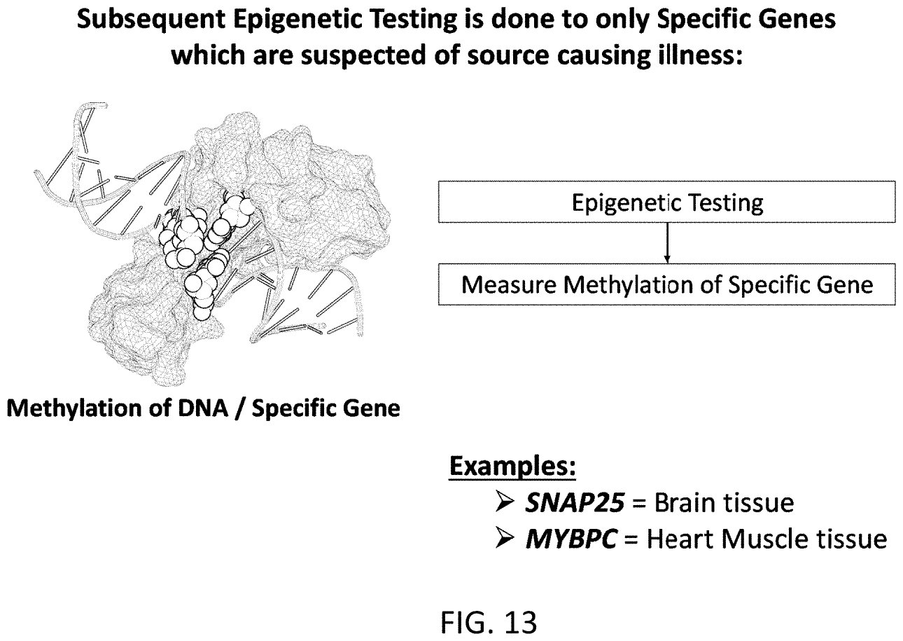

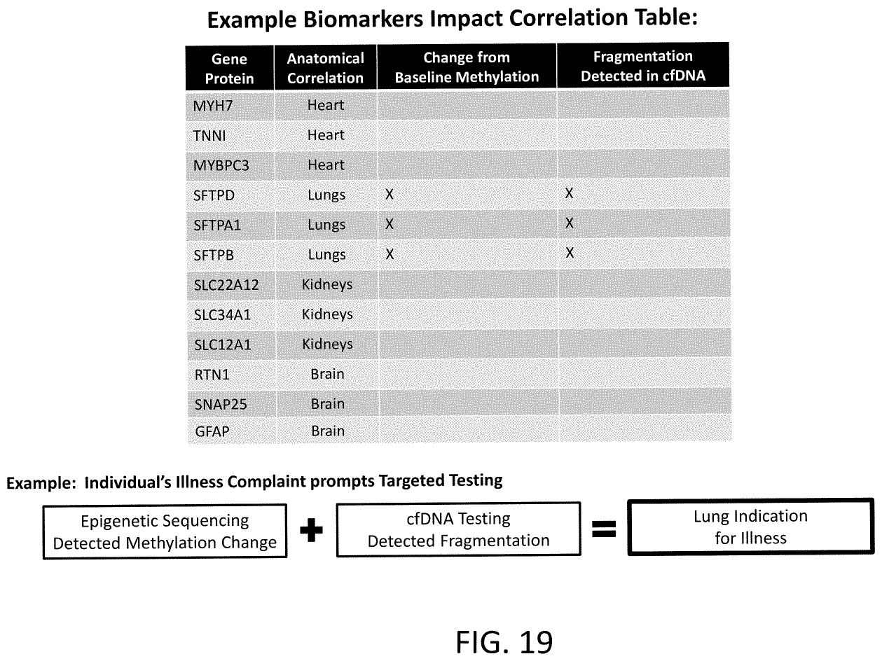

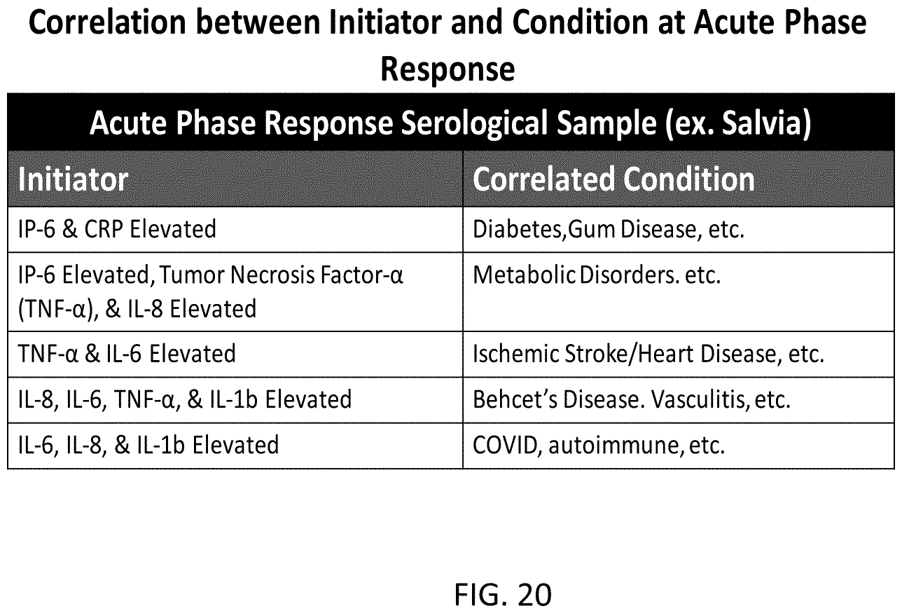

Although FIG. 18 illustrates exemplary biomarker correlations for the purposes of explanation, particular biomarker correlations are contemplated herein for application by the physiological assessment system 102. An example of particular biomarker correlations are illustrated in FIG. 20 with respect to testing using saliva. As an additional example, FIG. 13 illustrates the use of the epigenetic sequencing described herein at modularized DNA regions to determine if the gene or genes are being methylated or expressed in an anomalous method revealing a potential source of illness. Thus, FIG. 13 illustrates how subsequent epigenetic testing is leveraged to target only specific genes which are suspected of being elemental to the source-causing illness, allowing for the measurement of Methylation of the specific genes. FIG. 19 provides an example to show the impact correlation with biomarkers, as well as how methylation change (detected via epigenetic sequencing) and fragmentation (detected via cfDNA testing) combine to indicate how an individual's illness complaint provided to the system 102 or medical provider can be correlated to a specific anatomical organ/system. FIG. 20 details examples of the relationship of the initiator to the correlated condition when testing a saliva sample.

Accordingly, biomarkers are initially cataloged for each individual monitored by the physiological assessment system 102 as BMS data 116 which can then be used to identify later-in-time changes therein representing the individual may have been exposed to an impact event. This allows the physiological assessment system 102 to derive a multitude of information which can be used in real-time for a variety of practical applications. For example, individual or group biomarker detection can enable the monitoring of global under-reported and under-researched diseases through analysis of individuals originating from or visiting highly infectious areas which have an altered BMS due to their exposure. Biomarker detection can also provide early-warning data of potential future pathogenic health complications to individuals so that preventative action can be taken to address such complications. Further, the physiological assessment system 102 can immediately provide an alert to an individual and/or other entity(s) via wearable devices 125, 126 or other devices 126, 127 of possible exposure or local impact events which allows the individual and/or other entity(s) to take steps which can slow or halt, in the example of pathogenic exposure, the spread of pathogens to themselves or the public at large. As explained further herein, BMS segmentation and the ability of the physiological assessment system 102 to efficiently computationally assess BMS changes is critical to such tracking by enabling faster analysis, reporting and treatment while requiring less memory than conventional methods.

It should be noted that one or more of the wearables 125, 126 can be configured to monitor an individual's physiology to provide streamlined identification of one or more biomarkers indicating a physiological impact event. Physiological impact events monitored by a wearable 125, 126 may arise out of a variety of circumstances including but not limited to the introduction to an individual of a pathogen, infection, injury, disease, radiation and/or RF signals. The biomarker-impact relationship is then cataloged by the physiological assessment system 102 in databases 134 and used to track and develop the individual's health profile such as their BMS. The physiological assessment system 102 can use such data to identify possible health complications for the individual by, for example, reference to the biomarker impact correlation table 1800, as well as serve as a reference for developing and refining the biomarker impact correlation table 1800 with signs, symptoms, and conditions associated with the biomarker and physiological impact events.

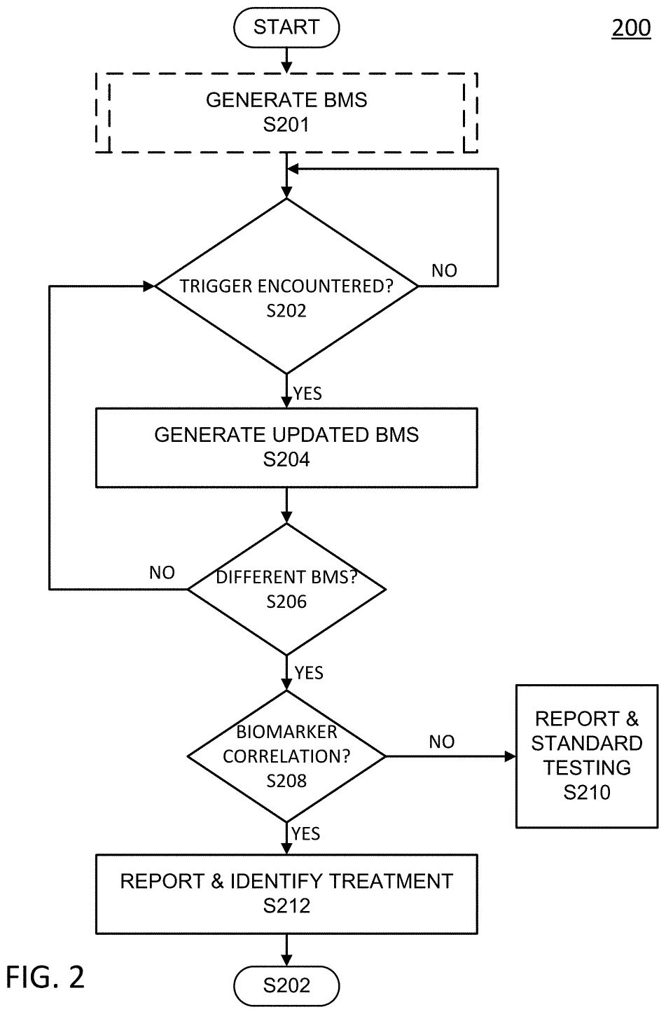

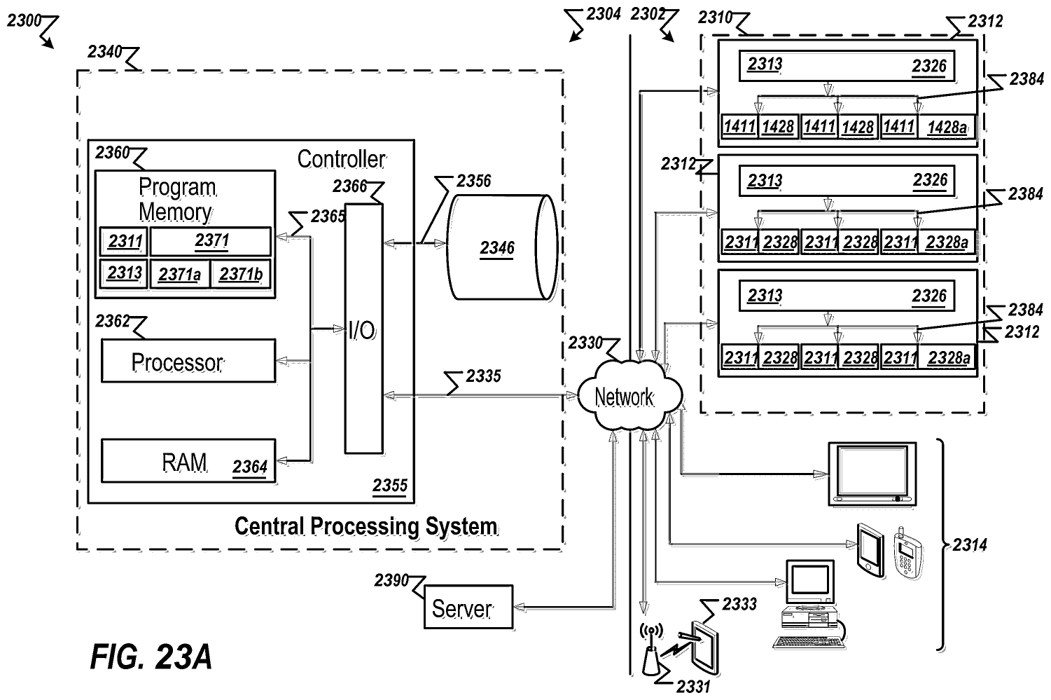

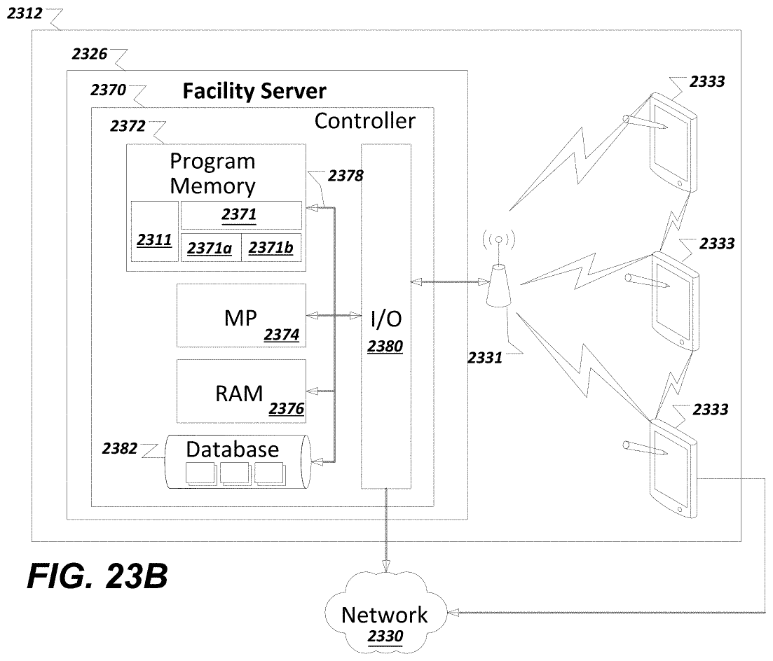

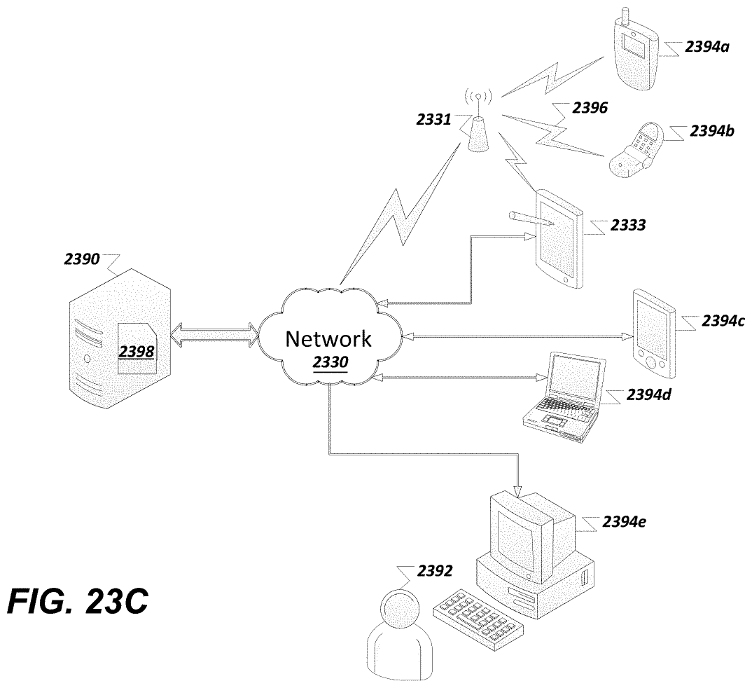

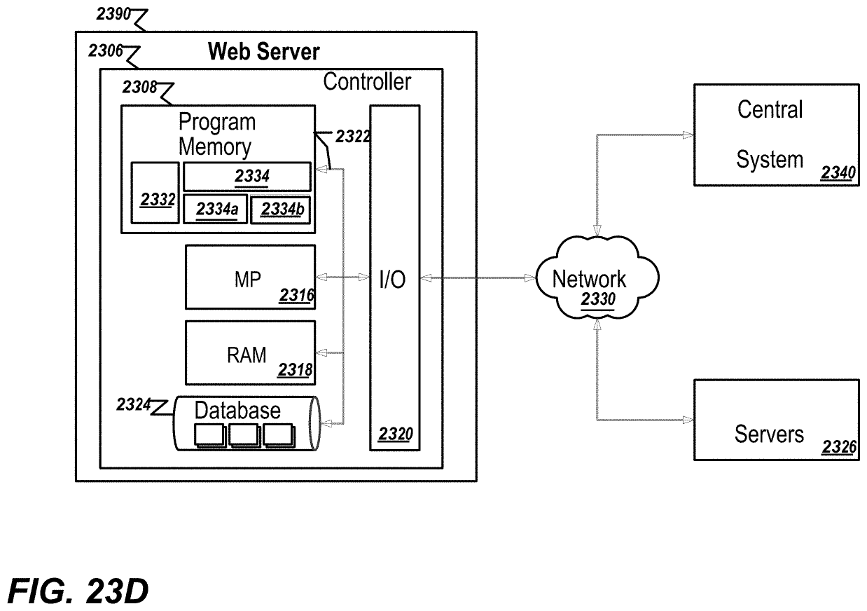

FIG. 2 is a flowchart illustrating an exemplary method 200 which can be implemented by the physiological assessment system 102 for the identification of changes in an individual's BMS that may be indicative of a physiological impact event resulting in a targeted tracking and/or treatment tailored to an individual. While the steps are presented in a particular order, those of skill in the art will recognize that the order of steps could be rearranged and/or some steps could occur at the same time without altering the nature of the method. The steps of the methods described herein are performed by physiological assessment system 102 engines using data in database 112 managed by the data management engine 104 from one or more of external databases 130 and internal databases 134 via servers 128 and 132, respectively, and one or more of devices 124-127. These engines can be implemented via hardware or software or a combination thereof and include the data management engine 104, the BMS engine 106, the data mining/collection engine 108, the treatment engine 109, the correlation engine 110 and the notification engine 111. Thus, the physiological assessment system 102 can include a combination of computer architecture and/or software for implementing the methods described herein via the engines at least with respect to the special purpose architectures and infrastructure described in FIGS. 23A-23D.

Initially, an individual is identified by the physiological assessment system 102 based on an activation by a user or third-party operator via one or more of the devices 124-127 or by the physiological assessment system 102 itself. For example, a user of the physiological assessment system 102 may login and have the physiological assessment system 102 track the user's individual health or a third-party operator (primary care physician) may manually activate the features of the physiological assessment system 102 regarding another individual having an individual medical profile 113 to track the individual's health. A third-party operator may also activate the physiological assessment system 102 to enable bio-surveillance tracking of multiple individuals to identify potential geographic pathogenic or other exposure activity. Alternatively, physiological assessment system 102 itself may be continuously running or activate automatically based on a triggering event it receives from one or more of the devices 124-127 and/or databases 130, 134 with respect to an individual. The individual can come from a group population or subpopulation of interest. The group population may be any type of group known to those of skill in the art, for example a herd, flock, or other collective group of animals. In some embodiments, the group population may be a commercial herd or flock on a farm or an endangered animal population.

The physiological assessment system 102 may be configured to identify any particular individual, such as by assigning the individual a system-generated identifier. Alternatively, or additionally, the physiological assessment system 102 may be configured to identify individuals based on their personal identification information (i.e. email, name social security number, birthdate, etc) and/or by a number such as a tag or organization designation, name, biological profile, or other identifying characteristic. Alternatively, or in addition, the physiological assessment system 102 may identify individuals based on biometric data received from wearables 125, 126. Accordingly, the individual may be a user of the physiological assessment system 102 interacting with the physiological assessment system 102 and/or may be an individual being remotely monitored by the physiological assessment system 102 based on the information received from one or more of the devices 124-127 and/or information populated in database 112 from databases 130 and/or 134. For example, as described further herein, GPS systems included within the devices 124-127 allow the physiological assessment system 102 to track individual location data 115, the alteration of which may trigger the physiological assessment system 102 to monitor a particular individual.

In this exemplary method, it is assumed in this example that an individual has been identified by the physiological assessment system 102. Optional Step 201 involves the data management engine 104 checking one or more of the databases 134 and/or 112 to determine if the individual is new to the physiological assessment system 102 or if the individual has an existing individual medical profile 113. If the individual is new to the physiological assessment system 102, the BMS engine 106 generates an individual medical profile 113 and stores that individual medical profile 113 in databases 134. Otherwise, processing proceeds to step 202 to check for triggers such as whether a predetermined amount of time has elapsed for individual monitoring or the individual has been exposed to an impact event.

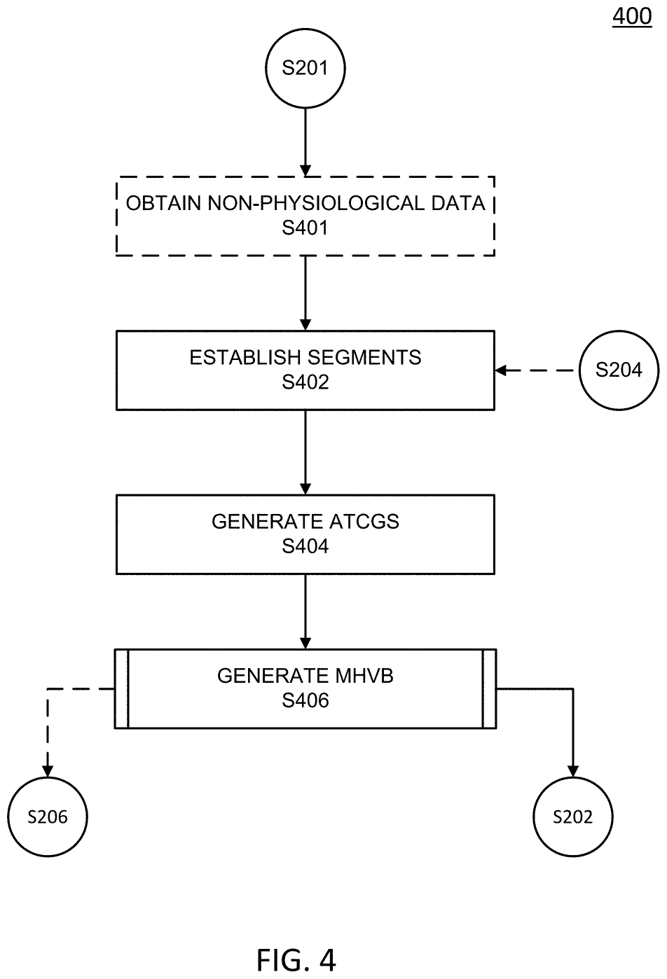

For individuals in which the physiological assessment system 102 does not yet have an individual medical profile 113, FIG. 4 illustrates a flowchart of an exemplary method for step 201 of generating the individual medical profile 113 including a BMS. Each identified individual will have a separate individual medical profile 113 having a variety of information in an individual medical profile 113 which, in one example, could be for an entire group of people or animals based on an amalgamation of associated data in databases 134 and/or 112. For the sake of simplicity, the methods and systems discussed herein are generally discussed only with respect to individual medical profile 113 although all actions are equally applicable to group medical profiles 113. For example, bio-surveillance methodologies described herein may involve the analysis of a plurality of individual medical profiles 113 or a joint medical profile 113 of a plurality of individuals generated as separate set of data.

The medical profiles 113 may include physiological data and non-physiological data. For example, at step 401 the data mining/collection engine 108 collects non-physiological biographical data 114 about the individual to populate the individual medical profile 113 with individual-specific data at which point the data management engine 104 stores the biographical data 114 in the databases 134. Data obtained at this step can include a variety of information including, but not limited to information relating to geographic origin, data of birth, personally identifiable information such as age and sex, family history, and/or medical history. The biographical data 114 may also include biological information about the individual that pertains to the individual's response to treatments for pathogens. Such information may include lung health information, such as respiratory capacity or the presence of tissue damage in the lungs, allergies, the individual's medical history of diseases and pathogens, group medical history such as family medical history or a history of exposures of the group to a pathogen, and/or a genetic propensity towards the development of certain conditions. The static biographical data 114 such as age and date of birth is added to the individual's baseline medical file. In one example, the dynamic data such as exposures and other treatment responses is appended to the individual's evolutionary history during routine check-ups or upon treatment for symptomatic illness by presentation of an individual at a clinician's site.

The biographical data 114 can be received from the devices 124-127 or may be obtained and/or supplemented over time by the data mining/collection engine 108 which can perform database and web crawling to obtain additional biographical data 114 about an individual from databases 130. Further, location data 115 can be collected and stored in the individual medical profile 113. This can include permanent location data such as the residence of the individual and real-time location tracking data managed by the data management engine 104 based on data received from devices 124-127. Accordingly, the data management engine 104 can continuously associate geographic information with an individual, such as a current location or recent geographical history such as travel locations. The location data 115 may be at any desired level of granularity, such as a continent, region, country, county, city, town, facility location, or even specific geographical coordinates. Environmental factors associated with the location data 115 may also be obtained by the data mining/collection engine 108 and/or devices 124-127, such as weather, climate, ambient pollutants, pathogens existing in the area, etc, and stored in databases 134 and/or 112. This data can be used by the physiological assessment system 102 as triggering data at step 202 to identify when an updated BMS should be generated for an individual. For example, exposure to ambient pollution or pathogens locally based on geographic travel of the individual can trigger the physiological assessment system 102 to perform an updated health assessment of the individual. Alternatively, other non-physiological biographical data 114 such as age and family medical history may be analyzed by AI/ML of the physiological assessment system 102 to trigger an updated health assessment of the individual if the individual's family is for example predisposed to certain conditions. Additionally, exposures documented by the physiological assessment system 102 to one individual can trigger updated health assessments of other family members or those co-located within a predetermined distance of the exposed individual. US Food and Drug Administration (FDA) recalls, market withdrawals, and safety alerts can be received by the physiological assessment system 100 from databases 130 by the data mining/collection engine 108 via servers 128 to cross check exposure(s) or provide personalized alerting of FDA warning activities. This data could be stored generally in database 134 for access and analysis by the BMS engine 106 and/or within the biographical data 114 as any particular information pertains to a specific individual.

Once the physiological assessment system 102 contains at least initial non-physiological identification information such as biographical data 114 and/or location data 115, the BMS engine 106 generates individual-specific physiological data as BMS data 116 and stores the BMS data 116 in databases 134. In one example, the BMS data 116 is generated via invasive or minimally invasive diagnostic tests such as blood counts (CBC and mean corpuscular hemoglobin (MCH), cerebrospinal fluid, tissue extraction, biopsies, respiratory pathogen panels, radiological testing, epigenetic testing, DNA, RNA and microRNA testing, or the like and associated analysis and/or cultures and/or noninvasive tests such as magnetic resonance imaging and neurological scans. These tests as performed result in the identification and mapping of a variety of biomarkers included in an individual's system which will establish the individual's BMS.

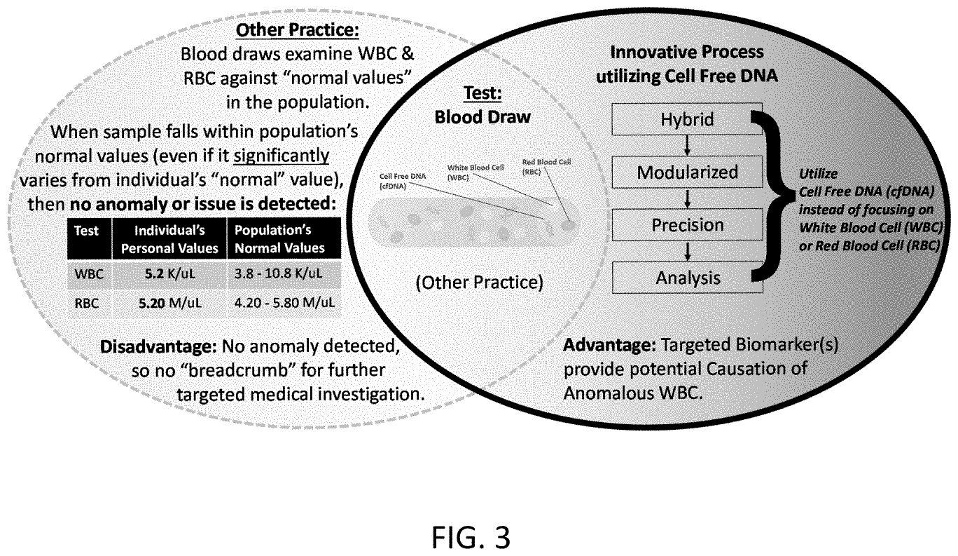

FIG. 3 illustrates a Venn diagram of physiological monitoring processes, including an exemplary process that utilizes cell free DNA (cfDNA) to leverage targeted biomarker(s) to provide potential causation of anomalous test results. As described in FIG. 3, the physiological assessment system 102 receives, via one or more of databases 130, 134 and/or devices 124-127, and utilizes information regarding several new assays and processes to serve as biomarkers for individual diagnosis as well as global bio-surveillance for new and emerging diseases.

In one example, an improved epigenetics process is implemented to measure methylation activities at a specific gene location to determine if an individual's gene activity and/or a pattern that the gene is expressed by or not expressed has changed since exposure (i.e. step 206). This process is enabled by the baseline assessment which uses gene pattern analysis to modularize organs and anatomic systems into segmented sequenced regions. If the gene(s) methylation process has changed, then additional testing is leveraged to confirm diagnosis of the suspected organ or anatomic system correlated to the gene. For example, if the SNAP25 gene has an anomalous methylation pattern, then additional testing of the brain is conducted to confirm the source of illness. Further novel testing patterns described herein are utilized to further isolate and confirm the specific organ or anatomic system that is the source of the illness. Second, the physiological assessment system 102 will receive, via one or more of databases 130, 134 and/or devices 124-127, and utilize novel assays for Cell Free DNA (cfDNA) which is isolated and extracted to provide an (immunoglobulin) Ig profile and subsequently measured for delta changes upon time/exposure/complaint of illness. Cells that have died from inflammation, disease, injury or other impact event release their DNA into the bloodstream referred to as cfDNA. Via novel assays, the dead cells can be detected and used to focus on the source organ or anatomic system that is releasing the diseased cells. Because organs and anatomic systems can be specifically identified in cfDNA based on a series of novel biomarkers as described herein, the testing, analysis and reporting can serve as bio-surveillance of the health of vital organs and anatomic systems. Third, processes for utilizing Locked Nucleic Acid (LNA) to create customized signatures for specific viruses, bacteria, fungi, protozoa, worms, parasites and other health exposure hazards can be compiled to a dynamic and constantly updated database 134 via the data mining/collection engine 108 for correlation by the BMS engine 106. This database 134 will serve as an online database for access by the physiological assessment system 102 to research and identify signatures of rare pathogens for diagnosis and for global bio-surveillance and pre-emptive warnings of new unidentified pathogens. Additionally, the physiological assessment system 102 can receive, via one or more of databases 130, 134 and/or devices 124-127, and utilize proteomic and major histocompatibility complex (MHC) research to further confirm diagnosis of suspected cause of illness. The physiological assessment system 102 receives, via one or more of databases 130, 134 and/or devices 124-127, and utilizes information regarding assays for proteomic and MHC.

In another example, genetic and epigenetic sequencing can be applied to establish the BMS data 116 as described below with respect to FIG. 4 via the physiological assessment system 102 performing the method 400 for establishing a BMS. Typically, applications of epigenetic sequencing include various methods, but all methods break the subject sample into deoxyribonucleic acid (DNA) markers and sequence through iterations of the DNA. The nucleotides found in DNA of (A) Adenine, (T) Thymine, (C) Cytosine, and (G) Guanine are cycled through and established in a long sequence data string of ATCG combinations. These approaches are very expensive and inefficient as they represent an all-in-one representation of a person.

To avoid these issues and provide a fast and memory-efficient mapping of DNA markers, the physiological assessment system 102 BMS engine 106 at step 402 breaks the individual down into a plurality of anatomical modular segments associated with anatomic systems of the individual body such as the cardiovascular system, respiratory system, gastrointestinal system, etc. In one example, molecular analysis of the RNA transcriptome from DNA transcription can be used to detect and map specific patterns of gene expression. References to already characterized genes can then be correlated to anatomic organs, tissues, and fluids. The process of molecular analysis to map RNA to organ, tissue, or fluids is improved by sectioning or dividing the sequencing into the modularized segments for subsequent baseline comparison testing. This modularized sequence can then be measured via epigenetic testing for methylation pattern changes upon subsequent health checks or hazardous exposures (i.e. step 206). A methylation change in which the gene is expressed or not expressed in a different pattern from a baseline can then be detected or biosurveilled for individual health maintenance or used as a global bio-surveillance tool for health monitoring (step 208 and 212).

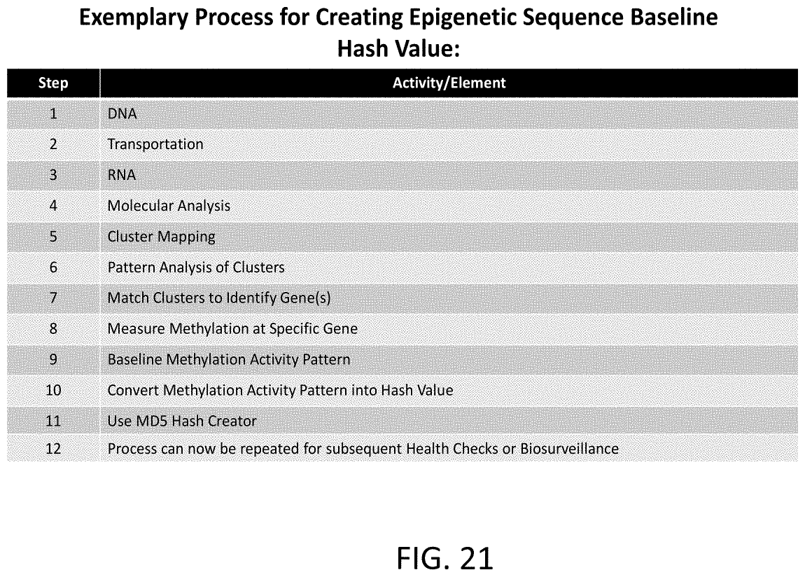

Thus, once the anatomical segments are identified at step 402, the BMS engine 106 generates at step 404 ATCG sequence data 120 for each of the identified segments and stores the ATCG data 120 in the databases 134 and 112. In other words, specific sequence profiles for each anatomic or organ system segment of the body are generated enabling long data sequences to be broken into modular groups. FIG. 21 illustrates an exemplary process for generating ATCG data 120 as steps 1-9 and as described herein.

Once the ATCGs have been generated for each modular anatomical segment at step 404, the BMS engine 106 converts the long data sequence ATCG data 120 format for each segment to mathematical hash values to derive a plurality of modular hash value baseline (MHVB) data 122 for each segment at step 306. FIG. 21 illustrates exemplary steps for creating the MHVB data 122 at steps 10 and 11. Accordingly, methylation can be measured and stored with respect to change related to how a gene currently expresses or does not express by activity pattern and the pattern can then be converted into a mathematical value as MHVB data 122 for later comparisons. The MHVB data 122 is then stored in databases 134 and 112 with respect to the corresponding individual profile 113. Accordingly, the BMS data 116 of each individual includes specific ATCG data 120 and MHVB data 122 for each corresponding segment of the individual.

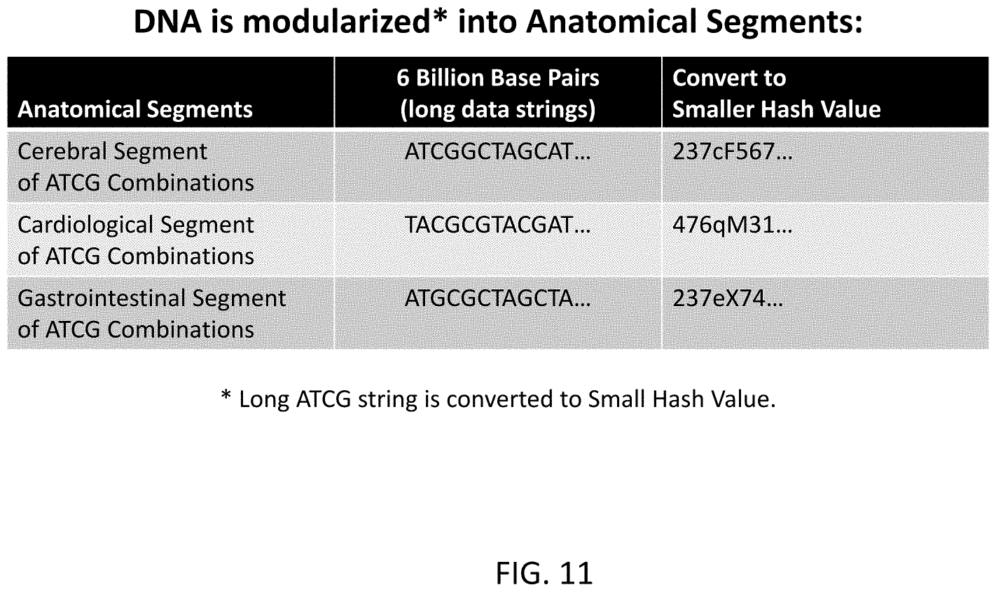

FIG. 11 illustrates an exemplary modularized breakdown of anatomical systems and corresponding representative MHVBs for each segment. Vice overcoming the technical constraints, limited applicability, and significant cost of using genetic (i.e., hereditary) testing, the processes for epigenetic sequencing described herein focus on efficient methodologies to identify and measure changes related to DNA expressions resulting from exposure(s)/impact events. FIG. 3 illustrates the need for the process and systems described herein by comparing (1) the limitations of the current practice of assessing White Blood Cell (WBC) and Red Blood Cell (RBC) which result from a blood draw against the normal value range for the population, with (2) the advantage of the systems and methods described herein that utilize Cell Free DNA (cfDNA) to leverage targeted biomarker(s) to provide potential causation of anomalous test results. This may be employed as often as necessary, such as for source detection when the individual complains about a symptomatic illness, or for routine surveillance check-ups.

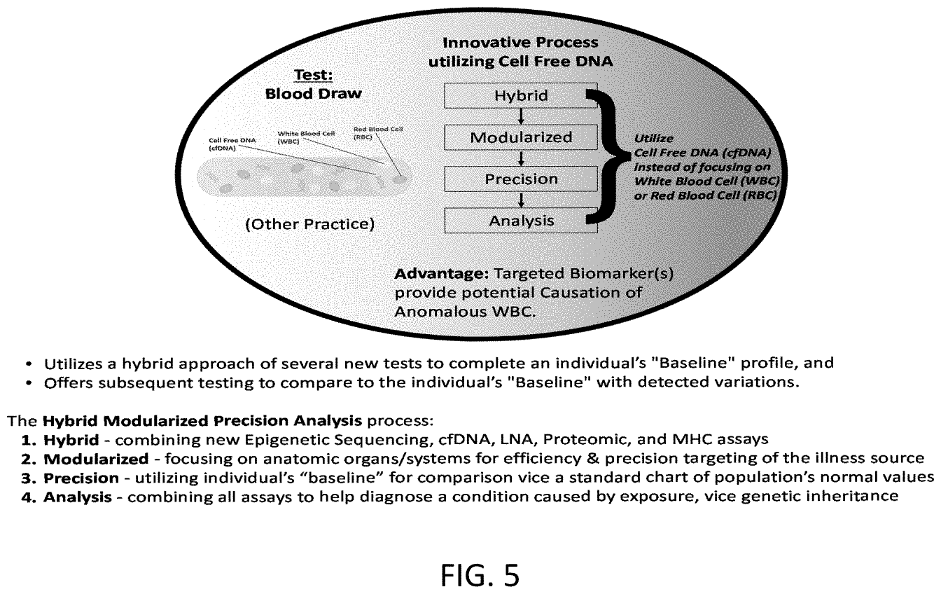

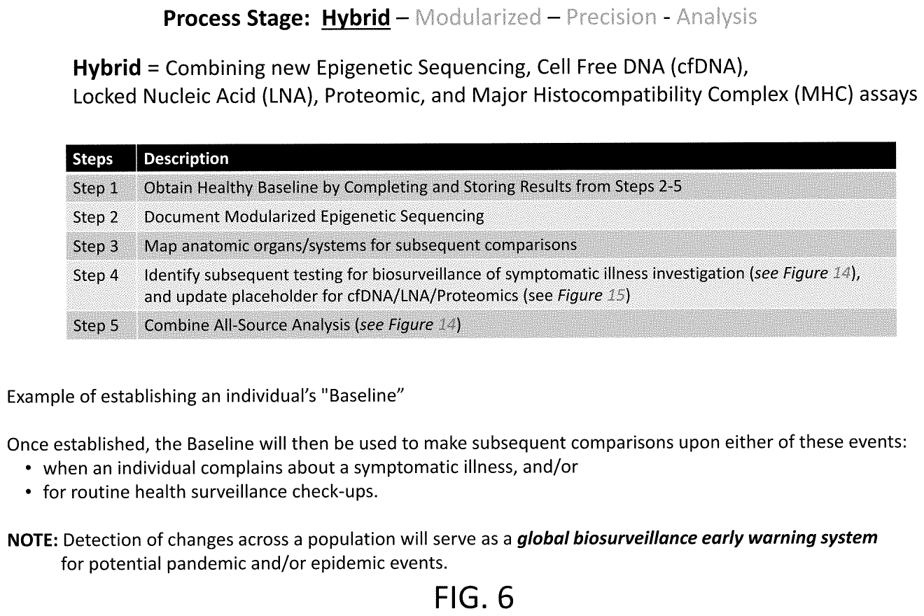

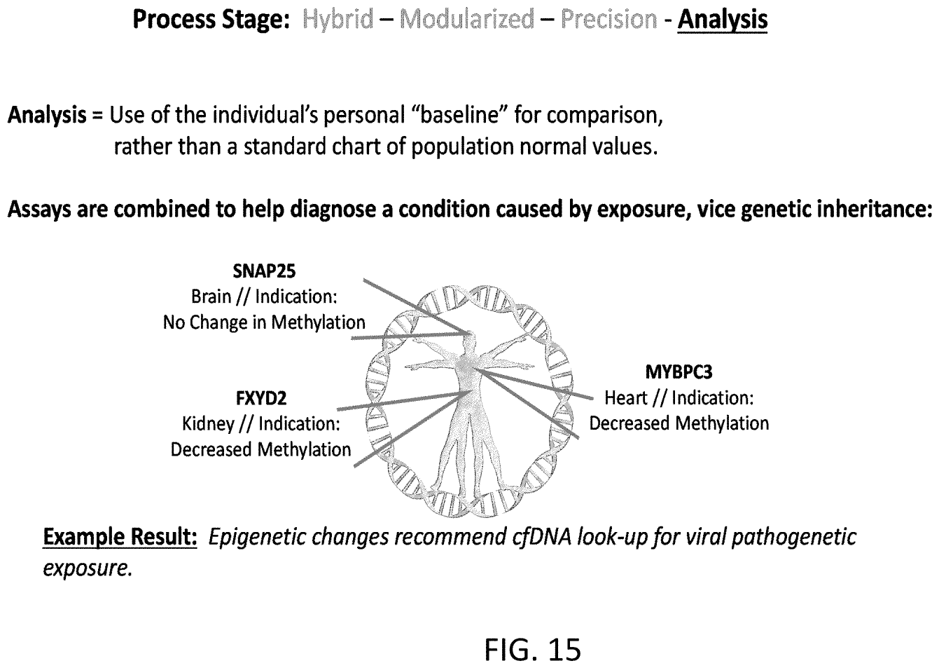

Additionally, the systems and methods described herein present the opportunity to detect novel changes across a population, as well as to serve as a global bio-surveillance early warning system for potential pandemic and/or epidemic events. FIG. 5 describes the Hybrid Modularized Precision Analysis stages that utilize several new tests to complete an individual's BMS in order to offer subsequent testing options to detect variations from the BMS. FIG. 5 illustrates how the physiological assessment system 100 leverages epigenetic testing (vice genomic, or hereditary, testing) to identify and to measure changes related to how the individual's DNA is expressed (i.e., altered) after the exposure(s). As such, the physiological assessment system 100 leverages “epigenetic” (vice “genomic”) testing to identify and to measure changes related to how the individual's DNA is expressed after exposures. FIG. 6 elaborates on the Hybrid aspect of this process, as it combines new epigenetic sequencing, cfDNA, LNA, Proteomic, and Major Histocompatibility Complex (MHC) assays through a 5 Step process. Thus, FIG. 6 elaborates on the “Hybrid” Process Stage of this novel technique, which combines new Epigenetic Sequencing, cfDNA, Locked Nucleic Acid (LNA), Proteomic, and MHC assays (FIG. 15) to establish an individual's health “Baseline” for targeted comparison(s) (FIG. 15) when an individual complains about a symptomatic illness to a medical provider or provides such information to the physiological assessment system 102 via devices 124-127 and/or for routine health surveillance check-ups, thus supporting both the needs of the individual as well as a global bio-surveillance early warning system for potential pandemic and/or epidemic events.

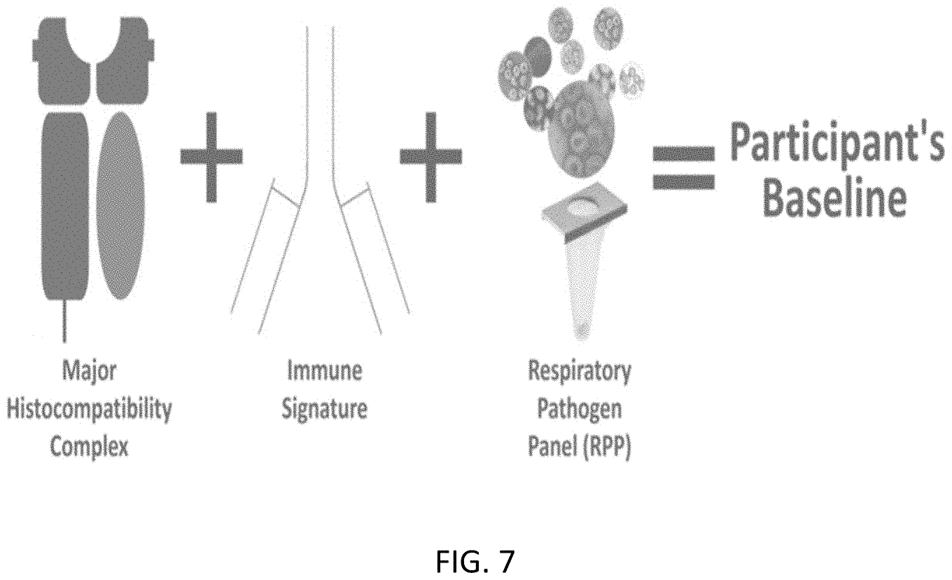

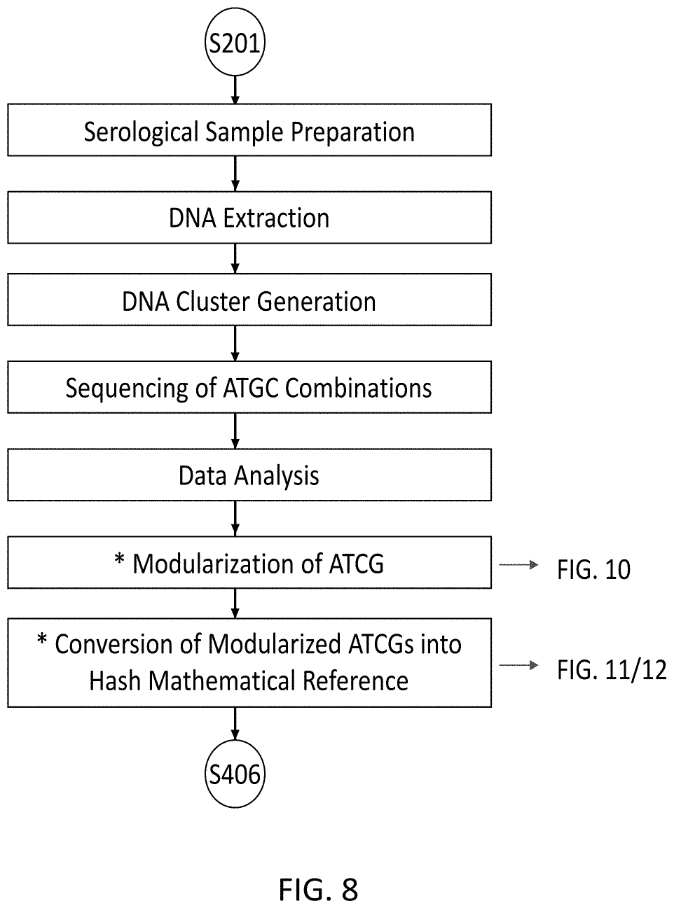

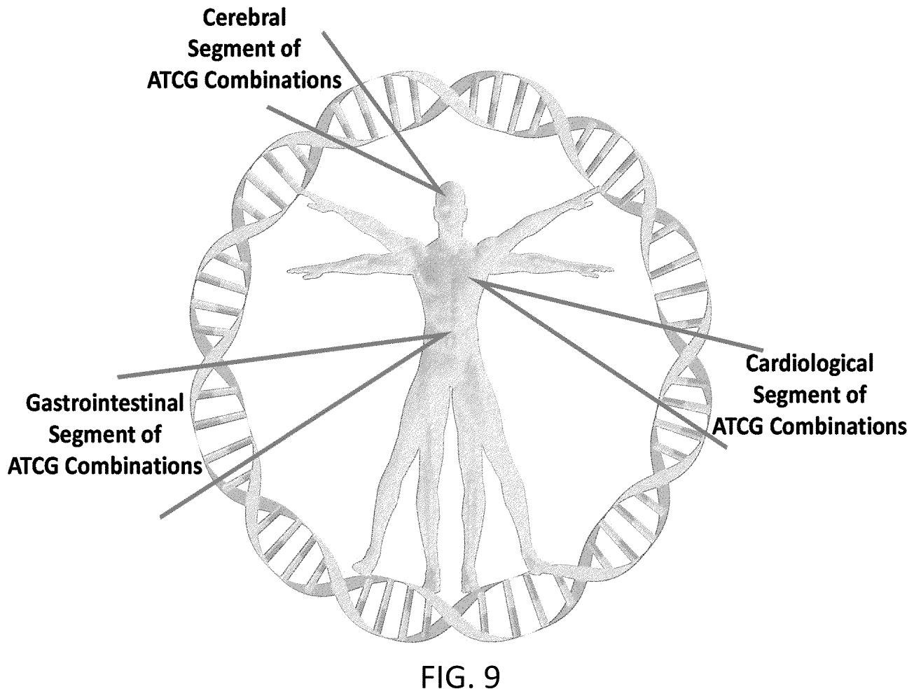

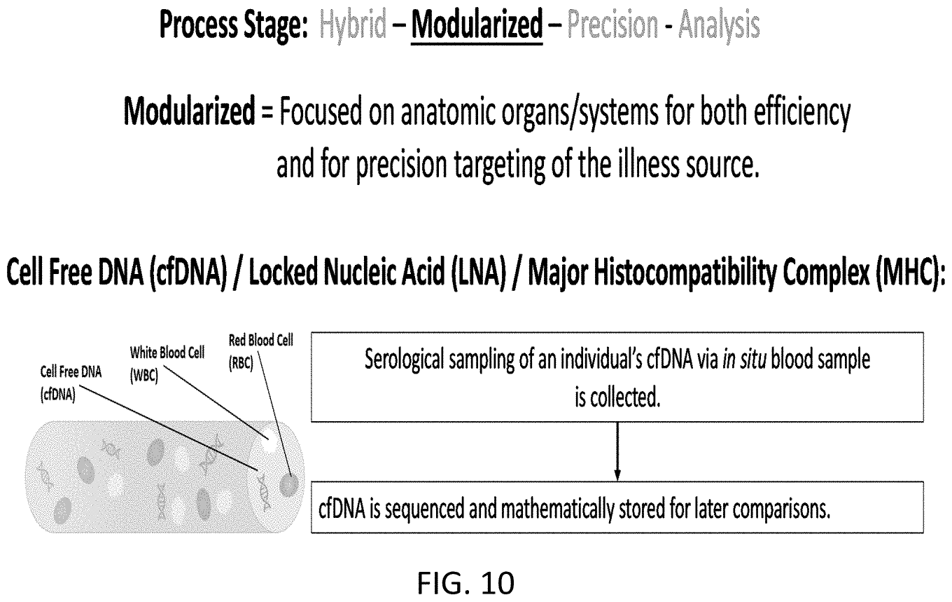

As an example, FIG. 7 illustrates that the BMS may be developed based on the individual's MHC, immune signature, and Respiratory Pathogen Panel (RPP). FIG. 8 illustrates a detailed workflow for the efficient process of epigenetic sequencing, resulting in the modularization of genetic code and subsequent conversion into hash mathematical references. The steps of preparation, extraction, cluster generation allow for the sequencing of ATCG combinations as in step 404 of FIG. 4 and illustrates that ATCG sequencing can be performed before or after segmentation as provided for in step 402 of FIG. 4 and noted in FIG. 8. Details of these processes are further described as indicated in FIGS. 10-12. FIG. 9 illustrates how modularized DNA relates to anatomical segments and FIG. 10 details the Modularized stage, which focuses on anatomic organs/systems for both efficiency and for precision targeting of these illness source. The Modularized aspect works from a serological sampling of an individual's cfDNA (obtained in one example from an in situ blood collection which could in one example be from wearable 125 and transmitted to the system 102), and then the sequenced results can be\stored in database 134 and/or 112. FIG. 11 provides an example of DNA that has been modularized, displaying anatomical segments (e.g., cerebral, cardiological, or gastrointestinal) with the long data strings (approximately 6 billion base pairs of ATCG) and converted references of smaller hash values.

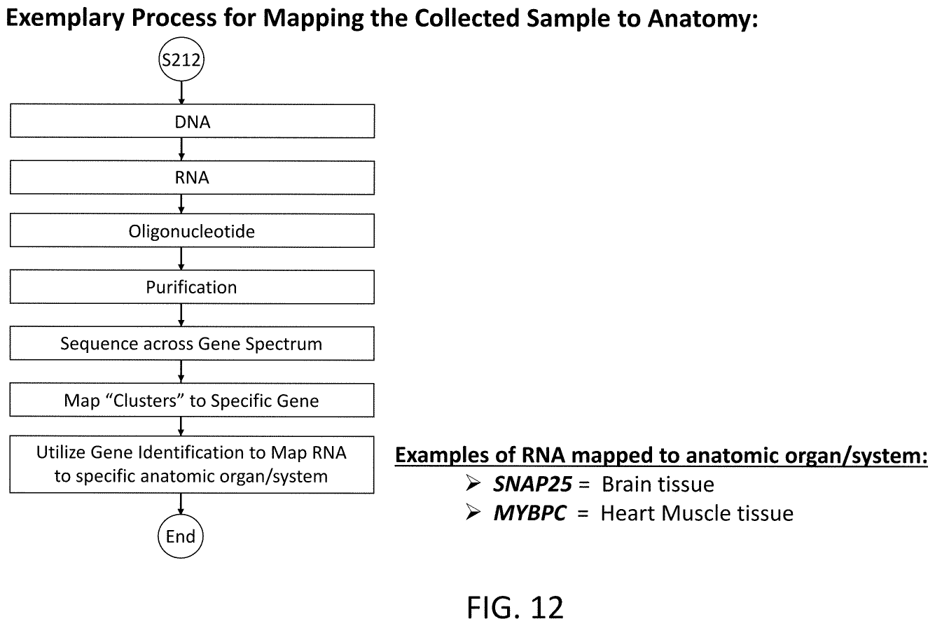

The process for mapping the collected sample to an anatomical segment is illustrated in FIG. 12, leveraging each sequence across the gene spectrum to map “Clusters” to the specific gene, and then utilizing gene identification to map RNA to the specific anatomic organ/system. The mapping serves as a baseline for an individual, allowing subsequent epigenetic testing to be performed for specific genes which may be the source which is causing the illness. FIG. 13 illustrates that the epigenetic testing serves to measure methylation of the specific gene, providing the indication of an exposure. The methylation DNA value is a component of the Precision aspect of the methodology, utilizing the individual's personal baseline for comparison—vice referring to a chart of the population's normal values.

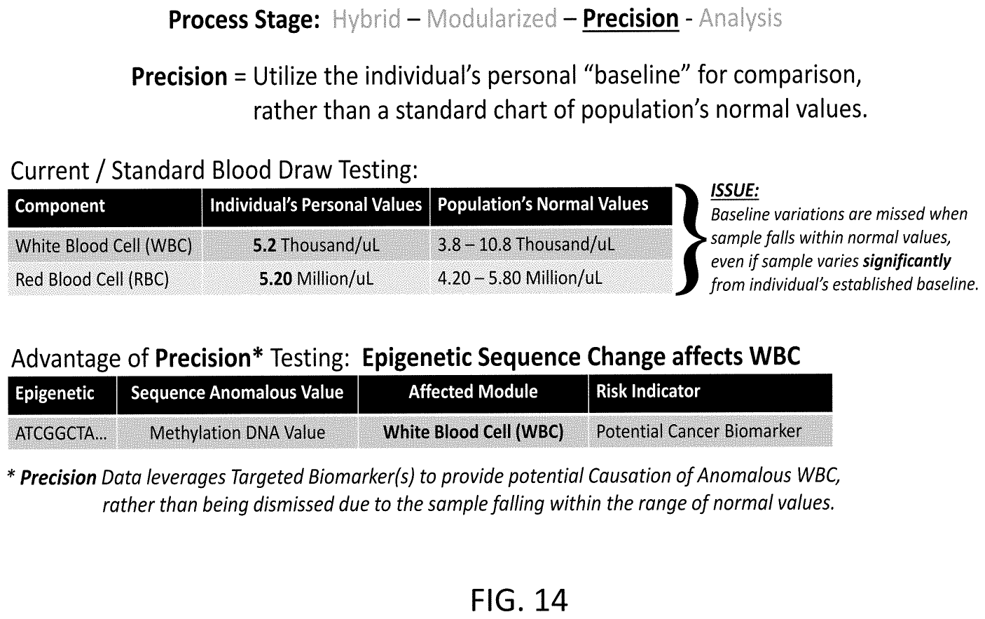

As FIG. 14 explains, Precision data leverages targeted biomarker(s) to provide potential Causation of Anomalous white blood cell (WBC), rather than having the WBC count dismissed as an indicator due to the sample falling within the range of normal values within a population. FIG. 15 illustrates another aspect of the methodology: Analysis, during which the individual's personal baseline is compared to the Precision data by combining all assays to help diagnose a condition caused by exposure (i.e. steps 206, 208). Thus, the systems and methods described herein leverage targeted biomarker(s) to provide potential causation of anomalous WBC, rather than risking missing an indicator because other blood draw tests only verify if the sample falls within the accepted range of normal values within the population.

The BMS data 116 represents a baseline immunity profile for the individual among other health and biological characteristics of the individual. The BMS data 116 may include information related to the presence or absence of various immunological biomarkers, such as immunoglobulins, antibodies, and the like. Other biomarkers may include information related to red blood cell counts, red blood cell size, white blood cell counts, and other blood-related indicators of infection or disease. Further, additional biomarkers may include information related to DNA or RNA that may indicate the presence of a foreign body such as a virus, bacteria, fungi, protozoa, or worm, or a type of cancer.

Once a BMS is established for the individual in step 201 or if one had already been established, the physiological assessment system 102 will at step 202 periodically update the individual's BMS data and/or monitor for any triggers indicating a potential change in the individual's medical status due for example to an impact event. Periodic updates triggered at step 202 may occur at any interval based on a dynamic BMS Update Timer 117 or may be manually requested by users of the physiological assessment system 102. Further, the periodicity of the updates, or BMS update time 117, may change over time as more biographical data 114 about the individual is acquired and/or the individual's activities increase or decrease in risk exposure as monitored by the physiological assessment system 102 via information obtained via the devices 124-127 and/or external databases 130 via periodic updates by the data mining/collection engine 108. For example, if the BMS engine 106 identifies based on the location data 115 that the individual has moved to an area with increased pathogenic activity, the BMS update timer 117 may be updated to reflect more frequent updates to the BMS data 116. Further, the BMS update timer 117 setting may be set to more frequent BMS data 116 updates for older individuals and/or individuals with specific medical histories. Therefore, each individual in the system with a medical profile 113 may have different established update periods by the BMS engine 106 based on the particular biographical data 114 contained in their medical profile 113. Further, the timelines for BMS updates may change throughout the lifetime of each individual. Accordingly, at step 202 if enough time has passed since the last update based on the setting of the BMS update timer 117, the BMS update engine 106 will generate updated BMS data 118 at step 204 using any of the methodologies described herein as discussed with respect to step 201.

Updates to the BMS data 116 by the BMS engine 106 may also be initiated at step 202 based on other triggering events such as impact events. For example, updated BMS data 118 may be generated as an individual moves from one geographic location to another based on monitoring by the BMS engine 106 of location data 115 particularly if the individual visits or passes through a region of increased pathogen exposure risk. This could include coverage of migratory animals, transportation of herd animals from one location to another, and people traveling. In some embodiments, the location data 115 may include environmental information associated with the location history of the individual or the population. Additionally, the physiological assessment system 102 may flag that the individual has come into contact with other individuals that have been exposed to pathogens or other harmful elements based on data collected by the data mining/collection engine 108 from databases 130 and/or data received via the data management engine 104 from devices 124-127 of other individuals tracked by the physiological monitoring system 102. Additional triggering events include physical injury to the individual as reported by the individual via the devices 124-127 or as detected by devices such as the wearables 125, 126. For example, the wearable 125, 126 accelerometers may indicate if the user has fallen and experienced physical injury or if there is an irregular heartbeat, body temperature or other such condition monitored by smart wearables. Further, biomolecular data obtained from wearable 125 from, for example, the sweat or blood of an individual may be processed by the wearable 125, or transmitted to the physiological assessment system 102 for processing, indicating that an updated BMS of the individual should be generated. Other impact events would similarly trigger the generation of an updated BMS such as exposure to radiation or RF signals as detected by wearable devices 125,126 and/or based on data retrieved by the data collection/mining engine 108 from databases 130 such as news reports or other indicative data sources.

Accordingly, checks for updating an individual's BMS may occur for any length of time, for example, a short duration, an established longer duration, or for the lifetime of the individual. For example, the checks may occur only when the individual is moving or being moved, such as the transportation of a herd of cattle from a ranch to a point of sale. In another example, the checks may occur over several years as a migratory individual moves over time or an individual makes routine visits to areas having one or more pathogens. The checks may occur for a predetermined number of years to track the pathogen exposure of an individual over the course of a lifetime or for generations of individuals, such as in a herd. If the physiological assessment system 102 databases 134 contain multiple individual medical profiles 113, the periodicity of checks and duration of checks may be the same for all individuals or may differ for unique individuals.

The physiological assessment system 102 can determine whether a change in medical status has taken place in one example by generating updated BMS data 118 of the individual, storing it in databases 134 and 112 and comparing it to the existing BMS data 116. The trigger to generate new BMS data 118 can be manually applied by the individual themselves or another administrative user of the physiological assessment system 102. The trigger can also be periodic based on a timeframe set by the individual, an administrator or by the physiological assessment system 102 itself based on the biographical data 114 of the individual such as age and/or medical history. The trigger to generate new BMS data 118 may also be applied based on impact event data such as location data 115 of the individual analyzed by the physiological assessment system 102 with respect to changes in locations or locations of other co-located individuals monitored by the physiological assessment system 102 via their biographical data 114 or devices 124-127, or data pulled for non-users by the data mining/collection engine 108 from the database 130. Biomolecular data retrieved from the wearable devices 125, 126 of the individual can also result in a trigger being generated and transmitted to the BMS engine 106. If there is a triggering event encountered at step 202, the BMS engine will generate updated BMS data 118 at step 204 using any of the methodologies described herein as discussed with respect to step 201.

In some embodiments, all of the processes performed in step 201 are repeated in update step 204 or optional step 401 may be omitted, for example, to completely update the individual's BMS data. Additionally, updates may be manually inputted into the system to update the biographical data 114 as other routine medical information is obtained such as in other routine and periodic health screenings, for example an annual physical or medical evaluation.

In another example, based on an impact event detected at step 202, the BMS engine 106 may generate updated BMS data 118 only for a specific part of the body based on the impact event detected at step 202. For example, the BMS engine 106 may generated updated BMS data 118 for a specific anatomical segment (i.e. brain) based on a particular impact event such as physical injury. In this case, updated ATCG sequence data 119 for this particular segment only is generated by the BMS engine 106 at step 404 which is stored in the database 134 and 112 with the corresponding updated BMS data 118. Once the particular ATCG segment data 119 has been generated for the particular anatomical segment, the BMS engine 106 converts the long data sequence ATCG data 119 format for this segment at step 306 to mathematical hash values to derive updated segment-specific MHVB data 121 as described herein with respect to step 406. The MHVB data 121 is then stored in databases 134 and 112 with respect to the corresponding individual profile 113. Accordingly, data records containing the updated BMS data 118 also include the updated ATCG data 119 and updated MHVB data 121 for the corresponding individual. In other examples, exposure to particular pathogens or other impact events as determined based on data from devices 124-127 and/or database 130 may trigger anatomic-specific BMS updates thereby saving processing time and complexity and allowing for quicker reports and alerts. Thus, based on data received from devices 124-127 and/or data management engine 104 via the data mining/collection engine 108, the BMS engine 106 can provide targeted BMS updates thereby supplementing particular BMS Data 118 segments. Once the updated BMS data 118 is generated, the process proceeds to step 206.

The updates to the BMS may be obtained at a specific location, such as in a veterinarian's office, a doctor's office, or a clinic, where physiological assessment system 102 may be updated directly, such as at a terminal 127 connected to the physiological assessment system 102 via a network such as the Internet. The updates may also be obtained where the individual is located, such as at a farm or a remote location, where the physiological assessment system 102 may be updated using a remote system connected thereto. In one example, the remote system may be one or both of the wearables 125, 126.

There is no limit to the number of updates to BMS data for use in physiological assessment system 102. Each update can create a unique record entry stored in databases 134. In this manner, health information at snapshots in time can be maintained for future use and study or analysis by AI/ML. In other embodiments, updated BMS data 118 may overwrite the existing BMS data 116, for example, such as to limit the amount of memory for any particular individual's records.

Once the BMS engine 106 has generated updated BMS data 118 it is determined by the BMS engine 106 at step 206 whether there are any differences in the BMS data 116 and updated BMS data 118. In one example, this involves the BMS engine 106 comparing the BMS biomarker data of the BMS data 116 and updated BMS data 118 to determine for example whether additional biomarkers exist or biomarkers from the BMS data 116 are absent in the updated BMS data 118. This can be a comparison of all of the biomarker data for each anatomical segment on record for the individual medical profile 113 or particular anatomically segmented data based on the particular impact event detected to reduce processing time and memory requirements. Alternatively, or in addition and based on the medical history of the individual, particular anatomical segments may be set by the BMS Engine 106 to always be updated and checked such as the brain for individuals with Alzheimers. The BMS engine 106 can also determine that a pattern of changes in the health profile 113 of an individual may indicate the onset of a disease, even if each unique change in the health profile is not alone a pathogenic biomarker. The BMS engine 106 may also be configured to assess similar changes across a plurality of individuals in a specific geographic region so as to predict an outbreak or the start of a pandemic. Further, any change to the BMS data 116 of one individual may create a cascade of requested updates and comparisons for other individuals based on a variety of information included in their medical profile 113 such as current or former proximity via their location data 115 to the flagged individual and/or family tree and history data for relatives of the individual. In another example, triggering events for one individual in a group of a plurality of individuals being tracked can result in requested updated BMS readings and analysis for the entire group which can provide information with respect to pathogenic spread and containment options.

If a particular anatomical segment was updated, the BMS engine 106 can compare the original MHVB data 122 for that particular segment to the MHVB data 121 included in the updated BMS data 118 to determine whether there are any differences. If there are no differences in the data, the updated BMS data 118 can be set to the current BMS data 116 (although prior BMS data 116 can still be maintained and stored in the databases 134) for future comparisons. At this point, the process proceeds back to stop 202 to restart the period for monitoring and/or to monitor for additional triggering events.

Otherwise, if the BMS engine 106 identifies one or more differences between the original BMS data 116 and updated BMS data 118 at step 206, the correlation engine 110 will analyze the differences with respect to the biomarker impact correlation table 1800 to determine whether a correlation can be found at step 208. For the purposes of discussion and in one example, FIG. 17 illustrates BMS data 116 which has been identified to have changed over time by the BMS engine 106 based on generated updated BMS data 118. For this example, it is assumed that the triggering event detected at step 204 may have been periodic or related to a specific impact event. It is also assumed that the BMS data 116 of FIG. 17 is from an initial generation by the BMS engine 106 and that biomarkers A, B and F are normally found in healthy individuals. However, based on a comparison of BMS data 116 to the updated BMS data 118, it is determined by the BMS engine 106 that the individual not only no longer has the biomarker F but they now have biomarkers E and G. Accordingly, at step 208 the correlation engine 110 refers to the biomarker impact correlation table 1800 illustrated in FIG. 18 to identify whether a correlation can be found based on the differences in BMS data.

The biomarker impact correlation table 1800 can contain a variety of associations between biomarkers and pathogens, diseases and other conditions. The biomarker impact correlation table 1800 may be any type of searchable data correlation system known to those of skill in the art. For example, the biomarker impact correlation table 1800 may be a ledger, a searchable and editable computerized data structure such as a spreadsheet, a database, or the like, or any other type of searchable data correlation system known to those of skill in the art. The data in biomarker correlation table 1800 may be sourced from any available source of medical information, such as from researchers, hospitals, clinics, universities, state, local, and federal governments, or the like. The biomarker impact correlation table 1800 can be updated over time manually as new correlations are discovered or automatically by the physiological assessment system 102 based on machine learning and artificially intelligent review of data included in databases 130 and 134 which provides for the monitoring of similar disease biomarkers in more than two individuals. For example, biomarker association data can be collected by the data mining/collection engine 108 from databases 130 for analysis by the physiological assessment system 102 to identify biomarker associations from newly published medical journals or other sources of such information. Further, AI/ML review of data within databases 130 of a variety of individuals by the physiological assessment system 102 may identify patterns in correlations between a variety of assessed biomarkers and patient medical conditions.

In the present example, a review of the updated BMS data 118 by the correlation engine 110 would identify that the individual may have been exposed to a specific pathogen as they no longer have biomarker F in their system. Also, the addition of biomarkers E and G may indicate an exposure to radiation. Accordingly, a biomarker correlation in this instance would be identified by the correlation engine 110 at step 208 and correlation data 123 specific to the aforementioned correlations would be stored in databases 134 and 112.

Once a correlation between a biomarker and a pathogen(s) and disorders (or other issues) is identified at step 208, the subsequently generated correlation data 123 may be used for a number of subsequent actions at step 212. For example, the treatment engine 109 can at step 212 prepare a medical report 131 of the correlation data 123, store the medical report 131 in the database 134 with the individual's medical profile 113 and the notification engine 111 can transmit the medical report 131 to the individual (or designated recipient such as a family member of primary care physician). The medical report 131 can be transmitted to any of the devices 124-127. The medical report 131 can also provide a description of targeted treatment options specific to the most recent BMS update. The targeted treatment options in one example could be based on a treatment correlation table identifying particular treatments for identified conditions or ailments at step 208. For example, the medical report 131 may provide treatment options specifically for addressing exposure to the aforementioned pathogen discovered in the biomarker impact correlation table 1800. It is further envisioned that for particular ailments or conditions identified by the correlation engine 110 that treatment instructions could be sent by the notification engine 111 to the wearable device 125 to prescribe certain treatments to the individual such as the release of certain chemicals or other reactants into the individual's system. For example, the wearable device 125 may be triggered via signals from the notification engine 111 to secrete potassium iodide to the individual based on exposure to radiation.

Further, as part of or separate from the reports, alerts may be sent to the individual, his medical provider or other entities based on the results of the biomarker correlation analysis at step 208. For example, the individual exposed to the pathogen in this scenario may have been traveling within an area having high levels of a disease thereby leading to the exposure. Based on the steps identified in FIG. 2, if it is determined by the BMS engine 106 that the individual has a different BMS based on a comparison of BMS data 116 and updated BMS data 118 which in turn has an identified biomarker correlation, the notification engine 111 can send an alert to the individual of their exposure along with steps for quarantining to prevent the additional spread of the disease. The notification engine 111 could also notify the individual's medical provider which could prepare the appropriate treatment. Further, local authorities where the individual lives based on the biographical data 114 and/or transportation authorities and other entities based on the current location data 115 of the individual could be notified by the notification engine 111 so that additional preventative steps may be taken to slow or halt the spread of the pathogen.

However, if at step 208 a biomarker correlation is not identified from the biomarker impact correlation table 1800 by the correlation engine 110, the notification engine 111 will put together a corresponding report 131 for transmission to the individual via devices 124-127 indicating the results of the correlation assessment and a recommendation to seek other testing at step 210 based on variations found in the BMS data. The report 131 can include the changes in biomarker makeup in the individual over time as well as indications of specific anatomical areas of the individual that the physiological assessment system 102 recommends testing via other methods. Any test results received via other methods can then be input either automatically or manually into the individual medical profile 113 to be stored in databases 134 for later retrieval and reference when performing the methodology described in FIG. 2. At this point, the process is completed and processing loops back to step 202 to continuously monitor an individual.

In other embodiments, a final step or one performed at any time might include research, such as using the data in the databases 134 to identify new correlations between biomarkers and pathogens, diseases, and/or medical conditions. In other aspects, the report 131 may not yield a specific treatment for a specific individual but could involve refining treatment approaches by adding information related to the response of classes of individuals to various treatments that can be used to target future treatments for individuals in those classes. Further, research could include studying the data in database 134 to understand the genesis or origin of a disease outbreak by using the location data 115 history of the individual(s) medical profile 113. Additional research activities could identify biomarkers for individuals with unknown conditions so that novel pathogens may be more readily identified and characterized.

As will be recognized by those in the art, the physiological assessment system 102 may be configured to both extract information from the databases 130, 134 and/or biomarker impact correlation table 1800 as well as to provide information back to either or both of the databases 134 and/or the biomarker impact correlation table 1800. For example, if physiological assessment system 102 were to diagnose an individual as having a particular condition, a new record entry in the individual medical profile 113 stored in databases 134 could be created and associated with the individual or the latest updated individual record entry for that individual could be updated automatically or upon a triggering event such as an approval by a doctor, technician, or researcher with the diagnosis. Similarly, if physiological assessment system 102 were to identify a new correlation between a biomarker and a pathogen, the biomarker correlation table 1800 could be updated automatically or upon a triggering event such as an approval by a doctor, technician, or researcher with the new information via a connection with the physiological assessment system 102.

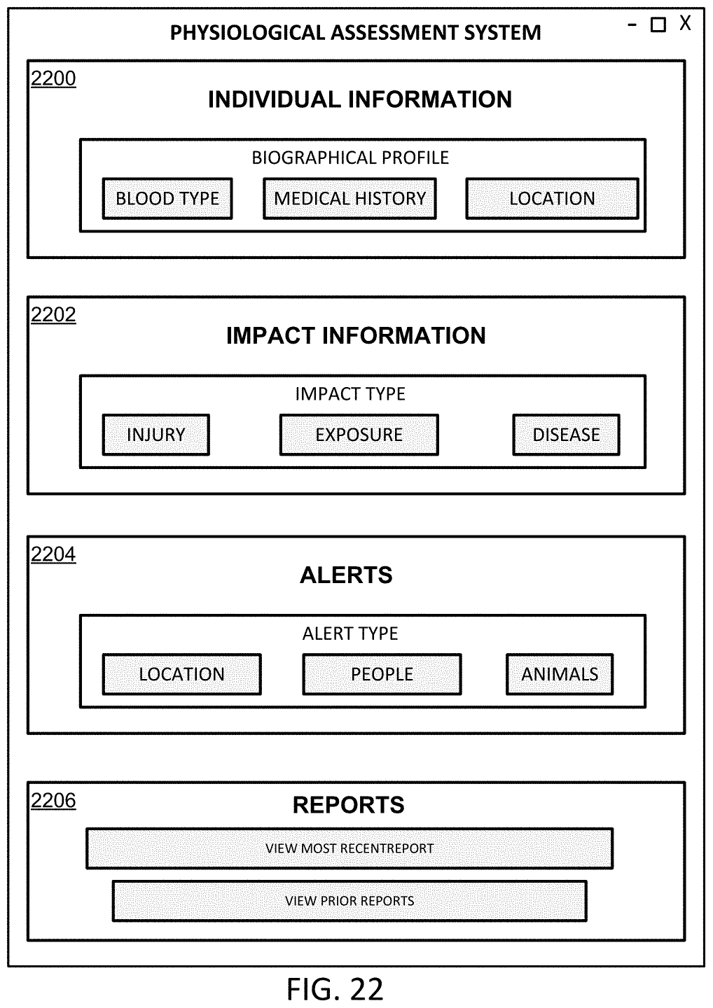

FIG. 22 illustrates an exemplary graphical user interface displayed on devices 124-127 for interaction with the physiological assessment system 102. In this example, a variety of windows 2200, 2202, 2204, and 2206 may be provided to the devices 124-127 to allow an individual or third party to interact with the system to report or receive information. For example, window 2200 relates to individual information and allows the individual user to update their medical profile 113 biographical data 114 by updating blood type, medical history, location data 115 and the like. Window 2202 allows the user to manually report impact event information that would result in a triggering a BMS update such as injury, exposure to pathogens, the development of a disease and the like. Window 2204 may provide the individual with alerts based on what is being monitored in their account such as alerts with respect to their location, other people around them or animals near them which may provide exposure risk to pathogens. The alerts could also be later-in-time indicators that other individuals with whom the individual has had contact with have developed conditions which could result in impact events to the individual based on BMS processing by the physiological assessment system 102 of the other individuals. Window 2206 allows the individual to view the most recent report data 131 or historical report data 131 with respect to their medical profile 113.

The examples discussed above with respect to windows 2200, 2202, 2204, and 2206 are intended to provide an overview of how an individual would interact with the physiological assessment system 102 and should not be construed as limiting the physiological assessment system 102 to the data presented therein. Additional windows, fields, inputs, outputs and interactive features could be included as part of additional or alternative GUIs.

As described herein, the physiological assessment system 102 provides numerous advantages over existing systems and current methodologies. For instance, treatment options for individuals may be rapidly deployed because the physiological assessment system 102 can more quickly identify a condition for an individual due to the extensive and readily available and current data in impact correlation table 1800 and the updated health history of the individuals in databases 134. Further, the MHVB data represents a mathematical “sum value” of each anatomic segment and can be used for faster more targeted computational comparisons for health checks. Accordingly, anatomical segment-targeted MHVB data provides for lower storage requirements and faster computational comparisons as the BMS engine 106 only needs to compare simpler mathematical data from a particular anatomical segment based on informative analysis arising out of a review of the biomarker impact correlation table 1800 by the correlation engine 110. The MHVB data also anonymizes the data from the individual thereby providing increased security while also reducing existing exorbitant costs for formerly required full-body epigenetic analysis. Thus, taken together, the MHVB sequence is technically revolutionary in approach as future medical evaluations and subsequent comparisons to baseline health require only sequencing of anatomic segments that are the subject of the health search and only the mathematical values are needed for initial comparison. The entire technical comparison transaction can then be efficiently serviced through a cloud computing environment. Accordingly, the methods described herein improve the functioning of computational devices by providing a particular manner of organizing data thereby allowing for faster processing with less memory when analyzing individual medical profiles 113 with respect to changes in the medical status of an individual.

The physiological assessment system 102 also provides a variety of practical applications. For example, the physiological assessment system 102 could employ artificial intelligence and/or machine learning to identify patterns related to a pathogen or other medical condition in an individual's medical status that traditional testing protocols would take significant lengths of time to identify, with additional time needed to correlate to a pathogen, disease, or other medical condition through traditional means like differential diagnosis. Accordingly, using the aforementioned processing efficiencies, the physiological assessment system 102 provides a host of real-time bio-surveillance functionality such as allowing for the tracking of individuals moving to or from high-risk areas that may expose them to impact events. By enabling real-time efficient tracking of such exposures, steps can be taken in advance to halt or slow the progress of pathogen exposure. The physiological assessment system 102 can also provide for immediate exposure alerts via devices 124-127 thereby allowing for easier tracking of patient-zero metrics when isolating pathogen sources. En masse, the physiological assessment system 102 can provide group-wide or region-wide impact event metrics which could enable rapid shutdown procedures both at a government level and patient-specific level via devices 124-127 to reduce spread. Further, wearable device 125 may be automatically activated by the physiological assessment system 102 to provide immediate life-saving treatments to individuals based on impact events and BMS data analysis.

Further, the physiological assessment system 102 may be configured to continuously monitor, for example by AI/ML, databases 130 and 134 to search for newly detected biomarkers and associated data patterns. Utilizing the modularized health baseline assessment, a primary care physician is able to order an epigenetic sequence comparison of just the gastrointestinal sequence segment of the baseline—which is affordable and timely to conduct due to the limited scope of the test, versus ordering an entire whole-body re-sequence and comparison. Further, if correlations cannot be identified, the BMS will provide low-cost and efficient analysis assays for clinicians to order for diagnostic purposes. The physiological assessment system 102 will first determine which major body category needs to be assessed, then determine which diagnostic section a physician should focus on from antibodies, epigenetics, or microbiome. For example, an individual exhibiting flu-like gastrointestinal symptoms that are negative indicated to existing flu tests, may warrant an antibodies and microbiome assessment of the gastrointestinal body category. Conversely, an individual with symptoms of neurologic anomalous conditions may be better-focused on epigenetic assays which are focused on the brain and neurological categories. A host of unique assays will be compiled from the available anatomic and categoric options tailored to presented conditions.

Additional practical applications include the physiological assessment system 102 allowing individuals to be alerted of lingering and/or latent physical effects even after they have seemingly recovered from an exposure as a result of the continuous BMS monitoring. Further, medical providers and other agencies will be alerted in real-time to individuals with certain conditions without needing the individual to seek medical attention as a result of the continuous BMS monitoring. Therefore, tracking infection timelines, the spread of the pathogen and treatment can be performed immediately. Additionally, the physiological assessment system 102 may provide specific treatment instructions which can be applied in real-time to the individual via their wearable 125.

FIGS. 23A and 23B illustrate various aspects of an exemplary architecture implementing a platform 2300 for monitoring for and identifying physiological impact events. As previously described, implementing the methodologies described here provides for an improvement in the functioning of the exemplary architecture by requiring less memory and allow for faster processing when monitoring and identifying physiological impact events. The high-level architecture includes both hardware and software applications, as well as various data communications channels for communicating data between the various hardware and software components. The platform 2300 may be roughly divided into front-end components 2302 and back-end components 2304. The front-end components 2302 are primarily disposed within a submitter or reviewer network 2310 including one or more submitters or reviewers 2312. The submitters or reviewers 2312 may be located, by way of example rather than limitation, in separate geographic locations from each other, including different areas of the same city, different cities, different states, or even different countries. The front-end components 2302 may include a number of workstations 2328. The workstations 2328, for example, can be local computers located in the various locations 2312 throughout the network 2310 and executing various applications for detecting image anomalies.

Web-enabled devices 2314 (e.g., personal computers, tablets, cellular phones, smart phones, web-enabled televisions, etc.) may be communicatively connected to locations 2312 and the system 2340 through a digital network 2330 or a wireless router 2331, as described below.

Referring now to FIG. 23A, the front-end components 2302, in some examples, include several facility servers 2326 disposed at the number of locations 2312 instead of, or in addition to, several workstations 2328. Each of the locations 2312 may include one or more facility servers 2326 that may facilitate communications between the web-enabled devices 2314 and the back-end components 2304 via a digital network 2330, described below, and between the terminals 2328, 2328A of the locations 2312 via the digital network 2330, and may store information for several submitters/approvers/accounts. associated with each facility. Of course, a local digital network 2384 may also operatively connect each of the workstations 2328 to the facility server 2326. Unless otherwise indicated, any discussion of the workstations 2328 also refers to the facility servers 2326, and vice versa. Moreover, environments other than the locations 2312 may employ the workstations 2328, the web-enabled devices 2314, and the servers 2326. As used herein, the term “location” refers to any of these points of contact (e.g., call centers, kiosks, Internet interface terminals, etc.) in addition to the locations 2312, etc. described above.

The front-end components 2302 communicate with the back-end components 2304 via the digital network 2330. One or more of the front-end components 2302 may be excluded from communication with the back-end components 2304 by configuration or by limiting access due to security concerns. For example, the web enabled devices 2314 may be excluded from direct access to the back-end components 2304. In some examples, the locations 2312 may communicate with the back-end components via the digital network 2330. In other examples, the locations 2312 and web-enabled devices 2314 may communicate with the back-end components 2304 via the same digital network 2330, but digital access rights, IP masking, and other network configurations may deny access of the web-enabled devices 2314. The web-enabled devices may also connect to the network 2330 via an encrypted, wireless router 2331.

The digital network 2330 may be a proprietary network, a secure public Internet, a virtual private network or some other type of network, such as dedicated access lines, telephone lines, satellite links and/or combinations of these. Where the digital network 2330 includes the Internet, data communication may take place over the digital network 2330 via an Internet communication protocol. In addition to one or more web servers 2390 (described below), the back-end components 2304 may include a central processing system 2340 within a central processing facility. The locations 2312 may be communicatively connected to different back-end components 2304 having one or more functions or capabilities that are similar to the central processing system 2340. The central processing system 2340 may include processing circuitry (e.g. one or more computer processors) 2362 adapted and configured to execute various software applications and components of the platform 2300, in addition to other software applications, such as a physiological assessments applications.

The central processing system 2340, in some embodiments, further includes a database 2346 (which may include one or more databases). The database 2346 can be adapted to store data related to the operation of the platform 2300. The central processing system 2340 may access data stored in the database 2346 when executing various functions and tasks associated with the operation of the platform 2300.

Although the platform 2300 is shown to include a central processing system 2340 in communication with three locations 2312, and various web-enabled devices 2314, different numbers of processing systems, locations and devices may be utilized. For example, the digital network 2330 (or other digital networks, not shown) may interconnect the platform 2300 to a number of included central processing systems 2340, hundreds of locations 2312, and thousands of web-enabled devices 2314. According to the disclosed example, this configuration may provide several advantages, such as, for example, enabling near real-time uploads and downloads of information as well as period uploads and downloads of information. This provides for a primary backup of all the information generated in the wireless data transfer process. Alternatively, some of the locations 2312 may store data locally on the facility server 2326 and/or the workstations 2328.

FIG. 23A also depicts one possible embodiment of the central processing system 2340. The central processing system 2340 may have a controller 2355 operatively connected to the database 2346 via a link 2356 connected to an input/output (I/O) circuit 2366. It should be noted that, while not shown, additional databases may be linked to the controller 2355, in place of and/or in addition to those discussed above and/or shown in the figures.

The controller 2355 includes a program memory 2360, the processing circuitry 2362 (a microcontroller or a microprocessor, for example), a random-access memory (RAM) 2364 and the I/O circuit 2366, all of which are interconnected via an address/data bus 2365. Although only one microprocessor 2362 is shown, the controller 2355 may include multiple microprocessors 2362. Similarly, the memory of the controller 2355 may include multiple RAMs 2364 and multiple program memories 2360. Although the I/O circuit 2366 is shown as a single block, it may include a number of different types of I/O circuits. The RAM(s) 2364 and the program memories 2360 may be implemented as semiconductor memories, magnetically readable memories and/or optically readable memories, for example. A link 2335 may operatively connect the controller 2355 to the digital network 2330 via the IO circuit 2366.

FIG. 23B depicts one possible embodiment of the front-end components 2302 located in one or more of the locations 2312. Although the following description addresses the design of the locations 2312, it should be understood by one of ordinary skill in the art that the design of one or more locations 2312 may be different from the design of other locations 2312. Also, each of the locations 2312 may have various different structures and methods of operation. One of ordinary skill in the art would also understand that while the embodiment shown in FIG. 23B illustrates some of the components and data connections that may be present in a location 2312, it does not illustrate all of the data connections that may be present in a location 2312. For exemplary purposes, one design of a location 2312 is described below but it should be understood that numerous other designs may be utilized.

Each of the locations 2312, as illustrated, has one or more portable computing devices 2333 (e.g, notebooks computers, tablet computers, smart phones, personal data assistants, etc.) and/or a facility sever 2326. The digital network 2384 and wireless router 2331 operatively connect the facility server 2326 to the number of portable computing devices 2333 and/or to other web-enabled devices 2314 and workstations 2328. The digital network 2330 may be a wide area network (WAN), a local area network (LAN), or any other type of digital network known to those of skill in the art. The digital network 2330 may operatively connect the facility server 2326, the portable computing devices 2333, the workstations 2328, and/or other web enabled devices 2314 to the central processing system 2340.

Each portable computing device 2333, workstation 2328, user device terminal 2328a or facility server 2326 includes a controller 2370 as depicted in FIG. 23B in relation to the server 2326. Similar to the controller 2355 of FIG. 23A, the controller 2370 includes a program memory 2372, processing circuitry (e.g. one or more microcontrollers or microprocessors) 2374, a random-access memory (RAM) 2376 and an input/output (I/O) circuit 2380, all of which are interconnected via an address/data bus 2378. In some examples, the controller 2370 may also include, or otherwise be communicatively connected to, a database 2382. The database 2382 (and/or the database 2346) includes data such as the data stored in the data repository 134 and/or 130 (FIG. 1). As discussed with reference to the controller 2355, it should be appreciated that although FIG. 23B depicts only one microprocessor 2374, the controller 2370 may include multiple microprocessors 2374. Similarly, the memory of the controller 2370 may include multiple RAMs 2376 and multiple program memories 2372. Although the FIG. 23B depicts the I/O circuit 2380 as a single block, the I/O circuit 2380 may include a number of different types of I/O circuits. The controller 2370 may implement the RAM(s) 2376 and the program memories 2372 as semiconductor memories, magnetically readable memories and/or optically readable memories, for example.

Either or both of the program memories 2360 and 2372 may also contain machine-readable instructions 2371 (i.e. software) for execution within the processing circuitry 2362 and 2374, respectively. The software 2371 may perform the various tasks associated with operation of the location or locations and may be a single module 2371 or a number of modules 2371a, 2371b. While the software 2371 is depicted in FIGS. 23A and 23B as including two modules, 2371a and 2371b, the software 2371 may include any number of modules accomplishing tasks related to location operation.

In addition to the controller 2370, the portable computing devices 2333, the workstations 2328 and the other web-enabled devices 2314 may further include a display and a keyboard as well as a variety of other input/output devices (not shown) such as a scanner, printer, mouse, touch screen, a track pad, track ball, isopoint, voice recognition system, digital camera, bar code scanner, RFID reader, etc. A user or administrator may sign on and occupy each portable computing device 2333, workstation 2328 or user device terminal 2328a using any available technique, such as entering a username and password. If a user signs on to the system 102 using a portable computing device 2333, the network 2384 communicates this information to the facility server 2326 so that the controller 2370 may identify which users are signed onto the platform 2300 and which portable computing device 2333, workstation 2327 or user device terminal 2328a the user is signed into.

Various software applications resident in the front-end components 2302 and the back-end components 2304 implement functions related to physiological assessment and provide various user interface means to allow users to access the platform 2300. One or more of the front-end components 2302 and/or the back-end components 2304 may include a user-interface application 2311 for allowing a user to input and view data associated with the platform 2300 and to interact with the platform as described herein. In one example, the user interface application 2311 is a web browser application and the facility server 2326 or the central processing system 2340 implements a server application 2313 for providing data to the user interface application 2311. However, the user interface application 2311 may be any type of interface, including a proprietary interface and may communicate with the facility server 2326 or the central processing system 2340 using any type of protocol including, but not limited to, file transfer protocol (FTP), telnet, hypertext-transfer protocol (HTTP) or other protocols known to those of skill in the art. Moreover, some embodiments may include the application 2311 running on the portable computing device 2333 in a location 2312. The central processing system 2340 and/or the facility server 2326 may implement any protocol compatible with the user-interface application 2311 running on the portable computing devices 2333, the workstations 2328, the web-enabled devices 2314 and adapted to the purpose of receiving and providing the necessary information during the data transfer process.

For purposes of implementing the platform 2300, the user interacts with location systems (e.g., the central processing system 2340) via a number of webpages. FIG. 23C depicts a web server 2390 connected via the network 2330 to a number of portable computing devices 2333 and other web-enabled devices through which a user 2392 may initiate and interact with the platform 2300. The web enabled devices may include, by way of example, a smart-phone 2394a, a web-enabled cell phone 2394b, a tablet computer 2333, a personal digital assistant (PDA) 2394c, a laptop computer 2394d, a desktop computer 2394e and other such devices. Any web-enabled device appropriately configured may interact with the platform 2300. The web-enabled devices 2333 and 2394 need not necessarily communicate with the network 2330 via a wired connection. In some instances, the web enabled devices 2333 and 2394 may communicate with the network 2330 via wireless signals 2396 and, in some instances, may communicate with the network 2330 via an intervening wireless or wired device 2331, which may be a wireless router, a wireless repeater, a base transceiver station of a mobile telephony provider or other such device. Each of the web-enabled devices 2333 and 2394 may interact with the web server 2390 to receive web pages, such as the web page 2398 depicted in FIG. 23C, for display on a display associated with the web-enabled device 2333 and 2394. It will be appreciated that although only one web server 2390 is depicted in FIG. 23C, multiple web servers 2390 may be provided for the purpose of distributing server load, serving different web pages and implementing different portions of the web interface.

Turning to FIG. 23D, the web server 2390, like the facility server 2326, includes a controller 2306. Similar to the controllers 2355 and 2370, the controller 2306 includes a program memory 2308, processing circuitry (e.g. one or more microcontrollers or microprocessors) 2316, a random access memory (RAM) 2318 and an input/output (I/O) circuit 2320, all of which are interconnected via an address data buss 2322. In some examples, the controller 2306 may also include, or otherwise be communicatively connected to, a database 2324 or other data storage mechanism (e.g., one ore more hard disk drives, optical storage drives, solid state storage drives or other such drive). The database 2324 may include data such as external source web profiles, product data, web page templates and/or web pages and other data necessary to interact with the user 2392 through the next network 2330. As discussed with reference to the controllers 2355 and 2370, it should be appreciated that although FIG. 23D only depicts one microprocessor 2316, the controller 2306 may include multiple microprocessors 2316. Similarly, the memory of the controller 2306 may include multiple RAMs 2318 and multiple program memories 2308. Although FIG. 23D depicts the I/O circuit 2320 as a single block, the I/O circuit 2320 may include a number of different types of I/O circuits. The controller 2306 may implement the RAM(s) 2318 and the program memories 2308 as semiconductor memories, magnetically readable memories, and/or optically readable memories, for example.

In addition to being connected through the network 2330 to the user devices 2333 and 2394, as depicted in FIG. 23C, FIG. 23D illustrates that the web server 2390 may also be connected through the network 2330 to the central processing system 2340 and/or one or more facility servers 2326. As described below, connection to the central processing system 2340 and/or to the one or more facility servers 2326 facilitates the platform 2300.

The program memory 2308 and/or the RAM 2318 may store various applications for execution by the processing circuitry 2316. For example, an application 2332 may provide a user interface to the server 2390, which user interface may, for example, allow a network administrator to configure, troubleshoot, or test various aspects of the server's operation, or otherwise to access information thereon. A server application 2334 operates to populate and transmit web pages to the web-enabled devices 2394, receive information from the user 2392 transmitted back to the server 2390, and forward appropriate data to the central processing system 2340 and the facility servers 2326, as described below. Like the software 2371, the server application 2334 may be a single module 2334 or a number of modules 2334a, 2334b. While the sever application 2334 is depicted in FIG. 23D as including two modules, 2334a and 2334b, the server application 2334 may include any number of modules accomplishing tasks related to implementation of the web server 2390. By way of example, the module 2334a may populate and transmit the web pages and/or may receive and evaluate inputs from the user 2392 to facilitate the wireless transfer of data from a first table to a second tablet, while the module 2334b may communicate with one or more of the back-end components to provide the requested data.

Typically, a user may launch or initiate a user interface application (e.g., a web browsers or other user application) from a web-enabled device, such as the web-enabled devices 2333 and 2394 to access the web server 2390 cooperating with the system 2340 to implement the platform 2300.

Obviously, numerous modifications and variations of the present subject matter are possible in light of the above teachings. It is therefore to be understood that within the scope of the appended claims, the present subject matter may be practiced otherwise than as specifically described herein.

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. The functions, processes and algorithms described herein may be performed in hardware or software executed by hardware, including computer processors and/or programmable circuits configured to execute program code and/or computer instructions to execute the functions, processes and algorithms described herein. Additionally, some implementations may be performed on modules or hardware not identical to those described. Accordingly, other implementations are within the scope that may be claimed.

The foregoing description, for purposes of explanation, used specific nomenclature to provide a thorough understanding of the present subject matter. However, it will be apparent to one skilled in the art that specific details are not required in order to practice the present subject matter. Thus, the foregoing descriptions of specific embodiments of the present subject matter are presented for purposes of illustration and description. They are not intended to be exhaustive or to limit the present subject matter to the precise forms disclosed; obviously, many modifications and variations are possible in view of the above teachings. The embodiments were chosen and described in order to best explain the principles of the present subject matter and its practical applications, they thereby enable others skilled in the art to best utilize the present subject matter and various embodiments with various modifications as are suited to the particular use contemplated. It is intended that the following claims and their equivalents define the scope of the present subject matter.

The descriptions of the various embodiments of the present subject matter have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and to enable others of ordinary skill in the art to understand the embodiments disclosed herein.

1. A method of identifying a disease comprising:
identifying an immune profile in an individual;
monitoring the individual for changes in the immune profile; and
correlating changes in the immune profile with a disease.

2. A system for predicting a disease comprising:
a storage medium configured with a database of biomarkers for an individual, including baseline entries of the biomarkers for the individual; and
a processor connected to the storage medium, wherein the processor is configured to compare the biomarkers to a correlation table of biomarkers and diseases.

3. The system of claim 2, wherein the database includes entries for multiple individuals.

4. The system of claim 2, wherein the processer is configured with an artificial intelligence and machine learning algorithm configured to monitor the database for similar disease biomarkers in more than two individuals.
